(19) United States
(12) Patent Application Publication
(10) Pub. No.: US 2011/0130710 A1
(43) Pub. Date: Jun. 2, 2011

(54) TREATMENT OF ABNORMAL OR EXCESSIVE SCARS

(76) Inventors: David Lawrence Becker, Abbots Langley (GB); Colin Richard Green, Epson (NZ); Bradford James Duft, Rancho Santafe, CA (US)

(21) Appl. No.: 12/809,916

(22) PCT Filed: Dec. 22, 2008
(86) PCT No.: PCT/US08/14028

§ 371 Date: Feb. 14, 2011

Related U.S. Application Data
(60) Provisional application No. 61/008,877, filed on Dec. 21, 2007.

(51) Int. Cl. A61K 31/7088; A61P 17/02; C12Q 1/68; A61B 17/3205; A61M 35/00

(52) U.S. Cl. 604/22; 514/44 R; 424/9.2; 514/44 A; 435/6.1

(57) ABSTRACT

Methods, compounds, compositions, kits and articles of manufacture comprising anti-connexin polynucleotides for prevention and/or treatment of abnormal scars, including keloid scars, hypertrophic scars, atrophic scars, and widespread scars.

This application is a National Stage Application under 35 U.S.C. §371 of International Application No. PCT/US2008/014028, filed on Dec. 22, 2008 which claims the benefit of priority to U.S. Provisional Application No. 61/008,877 filed on Dec. 21, 2007. The disclosures of both are incorporated herein by reference.

### FIELD

The inventions relate compositions and methods for treating, preventing and reducing abnormal or excessive scars, including keloid scars, hypertrophic scars, widespread (stretched) scars, and atrophic (depressed) scars, as well as formulations, articles and kits, and delivery devices comprising such compositions.

### BACKGROUND

The following includes information that may be useful in understanding the present invention. It is not an admission that any of the information provided herein is prior art, or relevant, to the presently described or claimed inventions, or that any publication or document that is specifically or implicitly referenced is prior art.

In humans and other mammals wound injury triggers an organized complex cascade of cellular and biochemical events that will in most cases result in a healed wound. An ideally healed wound is one that restores normal anatomical structure, function, and appearance at the cellular, tissue, organ, and organism levels. Wound healing, whether initiated by trauma, microbes or foreign materials, proceeds via a complex process encompassing a number of overlapping phases, including inflammation, epithelialization, angiogenesis and matrix deposition. Normally, these processes lead to a mature wound and a certain degree of scar formation. Although inflammation and repair mostly occur along a prescribed course, the sensitivity of the process is dependent on the balance of a variety of wound healing modulating factors, including for example, a network of regulatory cytokines and growth factors. Consequently, certain cytokines and growth factors have been reported as potential opportunities for therapeutic intervention to modulate the wound healing process.

Scars are the result of wounds that have healed, lesions due to diseases, or surgical operations. Hypertrophic and keloid scars occur when the tissue response is out of proportion to the amount of scar tissue required for normal repair and healing.

Certain regions of the body, including back, shoulders, sternum and earlobe, are especially prone to develop abnormal scars known as hypertrophic scars or keloids. These scars are bulky lesions representing an increased deposition of collagen fibers. They have the same clinical appearance: they are red, raised, and firm and possess a smooth, shiny surface. Whereas hypertrophic scars can flatten spontaneously in the course of one to several years, keloids persist and extend beyond the site of the original injury. As thickened red scars that exceed the boundary of an injury and may grow for a prolonged period of time, keloids are hyperplastic connective tissue masses that occur in the dermis and adjacent subcutaneous tissue, most commonly following trauma, in certain susceptible individuals. Keloid lesions are formed when local skin fibroblasts undergo vigorous hyperplasia and proliferation in response to local stimuli. The increase in scar size is due to deposition of increased amounts of collagen into the tissue. African-Americans are genetically prone to developing keloids. Keloid development has been associated with different types of skin injury including surgery, ear piercing, laceration, burns, vaccination or inflammatory process. Hypertrophic scars are masses which can result from burns or other injuries to the skin. Such scars are usually permanent and resistant to known methods of therapy. Patients suffering from hypertrophic scars or keloids complain about local pain, itchiness and local sensitivity, all of which compromise their quality of life as well as affect the individual body image.

Various therapies for keloids have had only limited success, and. Existing efforts to manage hypertrophic scars and keloids include surgery, mechanical pressure, steroids, x-ray irradiation and cryotherapy. Disadvantages have been reported to be associated with each of these methods. For example, surgical removal of the scar tissue may be often incomplete and can result in the development of hypertrophic scars and keloids at the incision and suture points, i.e., scarring frequently recurs after a keloid is surgically removed, and steroid treatments may be unpredictable and often result in depigmentation of the skin. Simple surgical excision of keloid scars has a 50%-80% risk of recurrence. A combination of surgery with either intralesional corticosteroid injection or radiotherapy has been the typical treatment. However, intralesional corticosteroid injection is prone to complications (fat atrophy, dermal thinning, and pigment changes).

Atrophic or depressed scars resulting from an inflammatory episode are characterized by contractions of the skin, and leave a cosmetically displeasing and permanent scar. The most common example is scarring which occurs following inflammatory acne or chickenpox. The depression occurs as a normal consequence of wound healing, and the scar tissue causing the depression is predominantly comprised of collagen resulting from fibroblast proliferation and metabolism. Some acne patients are successfully treated using steroids injected intralesionally, topical liquid nitrogen applications, or dermabrasion. In many cases, however, there is either no improvement or the treatment results in other complications. Additional disfiguring conditions of the skin, such as wrinkling, cellulite formation and neoplastic fibrosis also appear to result from excessive collagen deposition, which produces unwanted binding and distortion of normal tissue architecture. Collagenase, an enzyme which degrades collagen, has been injected intralesionally to reduce scarring in these conditions. However, multiple disfigurements may arise, which make local treatments difficult or impossible.

Widespread (stretched) scars appear when the fine lines of surgical scars gradually become stretched and widened, which usually happens in the three weeks after surgery. They are typically flat, pale, soft, symptomless scars often seen after knee or shoulder surgery. Stretch marks (abdominal striae) after pregnancy are variants of widespread scars in which there has been injury to the dermis and subcutaneous tissues but the epidermis is unbreached. There is no elevation, thickening, or nodularity in mature widespread scars, which distinguishes them from hypertrophic scars. Atrophic scars have been treated with chemical peels, cutaneous laser resurfacing, dermabrasion, punch excisions, and the use of soft tissue biological and alloplastic biological fillers.

Thus, despite advances in the understanding of the principles underlying the wound healing process, there remains a significant unmet need in suitable therapeutic options for the treatment and prevention of abnormal scarring, including keloid and hypertrophic scarring, atropic scarring, and widespread scarring. There is a need in the art for a method of treating conditions such as these that are caused by abnormal or excessive scar formation.

Gap junctions are cell membrane structures that facilitate direct cell-cell communication. A gap junction channel is formed of two connexons (hemichannels), each composed of six connexin subunits. Each hexameric connexon docks with a connexon in the opposing membrane to form a single gap junction. Gap junction channels are reported to be found throughout the body. Tissue such as the corneal epithelium, for example, has six to eight cell layers, yet is reported to expresses different gap junction channels in different layers with connexin 43 in, the basal layer and connexin 26 from the basal to middle wing cell layers. In general, connexins are a family of proteins, commonly named according to their molecular weight or classified on a phylogenetic basis into alpha, beta, and gamma subclasses. At least 20 human and 19 murine isoforms have been identified. Different tissues and cell types are reported to have characteristic patterns of connexin protein expression and tissues such as cornea have been shown to alter connexin protein expression pattern following injury or transplantation (Qui, C. et al., (2003) Current Biology, 13:1967-1703; Brander et al., (2004), J. Invest Dermatol. 122:1310-20).

Antisense technology has been reported for the modulation of the expression for genes implicated in viral, fungal and metabolic diseases. See, e.g., U.S. Pat. No. 5,166,195, (oligonucleotide inhibitors of HIV), U.S. Pat. No. 5,004,810 (oligomers for hybridizing to herpes simplex virus Vmw65 mRNA and inhibiting replication). See also U.S. Pat. No. 7,098,190 to Becker et al. (formulations comprising antisense nucleotides to connexins). Peptide inhibitors (including mimetic peptides) of gap junctions and hemichannels have been reported. See, e.g., Berthoud, V. M. et al., Am J. Physiol. Lung Cell Mol. Physiol. 279: L619-L622 (2000); Evans, W. H. and Boitano, S. Biochem. Soc. Trans. 29: 606-612, and De Vriese A. S., et al. Kidney Int. 61: 177-185 (2001). See also Becker and Green PCT/US06/04131 (“Anti-connexin agents and uses thereof”).

### BRIEF SUMMARY

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this Brief Summary, which is included for purposes of illustration only and not restriction.

The present invention relates to methods of using anti-connexin polynucleotides for the treatment and prevention of abnormal or excessive scarring, as well as excessive scar formation and other types of abnormal or excessive proliferation of tissue, including keloid scars, hypertrophic scars, widespread scars, and atrophic scars.

In one aspect, the invention relates to methods and compositions for preventing or decreasing abnormal or excessive scar formation by administering to a subject in need thereof an effective amount of an anti-connexin polynucleotide. In all methods and compositions, anti-connexin 43 polynucleotides are preferred.

Subjects to be treated include those having experienced trauma, surgical intervention, burns, and other types of injuries that lead, or can lead, to abnormal or excessive scarring. The anti-connexin polynucleotide is administered in an amount effective to prevent and/or decrease abnormal or excessive scarring, i.e. the formation of high density tissue including cells and connective tissue (including scars, keloid and/or hypertrophic scars, atrophic scars, and wide-spread scars), without preventing normal wound closure. The anti-connexin polynucleotide can be administered locally and/or topically, as needed. In one embodiment, the anti-connexin polynucleotide would typically be applied at the time of surgery, preferably in a topical, instillation, or controlled release formulation and/or using barrier technology.

The invention also relates to a method of treating a subject having a keloid, scar, a hypertrophic scar, an atrophic scar, a widespread scar which method comprises: (a) excising the keloid scar, a hypertrophic scar, atrophic scar, widespread scar to create a wound, and (b) administering an anti-connexin polynucleotide to the subject in a quantity sufficient to prevent or reduce keloid, hypertrophic, atrophic, or widespread scarring, at a site of the wound.

The invention also relates to a method of preventing or decreasing keloid formation in a subject abnormal or excessive scar formation, including formation of a keloid scar, a hypertrophic scar, an atrophic scar, a widespread scar, in a patient in need thereof or at risk thereof, said method comprising administering a therapeutically effective amount of an anti-connexin polynucleotide to said subject. In certain embodiments, the method of preventing or decreasing keloid formation in a subject abnormal or excessive scar formation, including keloid scar, hypertrophic scar, atrophic scar, widespread scar formation, in a patient in need thereof or at risk thereof, comprises administering a therapeutically effective amount of an anti-connexin oligonucleotide to said subject. In other embodiments, the method of preventing or decreasing keloid formation in a subject abnormal or excessive scar formation, including keloid scar, hypertrophic scar, atrophic scar, widespread scar, in a subject in need thereof or at risk thereof, comprises administering a therapeutically effective amount of an anti-connexin 43 polynucleotide peptide to said subject.

The invention also relates to a method of preventing or decreasing hypertrophic scar formation in a subject in need thereof or at risk thereof, said method comprising administering a therapeutically effective amount of an anti-connexin polynucleotide to said subject. In certain embodiments, the method of preventing or decreasing hypertrophic scar formation in a subject in need thereof or at risk thereof, comprises administering a therapeutically effective amount of an anti-connexin oligonucleotide to said subject. The invention further relates to a method of decreasing or preventing excessive scar formation which comprises administration to a subject in need of treatment an effective amount of an anti-connexin polynucleotide. In one embodiment, the anti-connexin polynucleotide decreases or prevents keloid formation. In certain embodiments, the keloid is associated with surgery. In other embodiments the keloid associated with surgery is associated with a surgical incision. In another embodiment, the anti-connexin polynucleotide decreases or prevents hypertrophic scar formation. In another embodiment, the polynucleotide is an oligonucleotide.

In certain embodiments, the keloid scar, hypertrophic scar, atrophic scar, widespread scar, or other abnormal or excessive scarring, is associated with surgery. In other embodiments the keloid scar, hypertrophic scar, atrophic scar, widespread scar, or other abnormal or excessive scarring, associated with surgery is associated with a surgical incision. In other embodiments, the keloid scar, hypertrophic scar, atrophic scar, widespread scar, or other abnormal or excessive scarring, is associated with trauma. In still other embodiments, the keloid scar, hypertrophic scar, atrophic scar, widespread scar, or other abnormal or excessive scarring, is associated with pregnancy or giving birth.

In certain embodiments, the anti-connexin polynucleotide decreases connexin protein expression, wherein said connexin is selected from the group consisting of connexin 26, connexin 30, connexin 30.3, connexin 31.1, connexin 32, connexin 36, connexin 37, connexin 40, connexin 40.1, connexin 43, connexin 45, connexin 46 and connexin 46.6. In a preferred embodiment, the anti-connexin polynucleotide decreases expression of connexin 43. In another preferred embodiment, the connexin is a human connexin. In another embodiment the connexin is an animal connexin. In still other embodiments, the animal connexin is a dog, cat, horse, pig, sheep or cow connexin.

Examples of a connexin antisense polynucleotide include, for example, an anti-connexin oligodeoxynucleotide (ODN), including antisense (including modified and unmodified backbone antisense; e.g., a DNA antisense polynucleotide that binds to a connexin mRNA), RNAi, and siRNA polynucleotides.

Suitable connexin antisense polynucleotides include for example, antisense ODNs against connexin 43 (Cx43), connexin 26 (Cx26), connexin 37 (Cx37), connexin 30 (Cx30), connexin 31.1 (Cx31.1) and connexin 32 (Cx32). In certain embodiments, suitable compositions include multiple connexin antisense polynucleotides in combination, including for example, polynucleotides targeting Cx 43, 26, 30, and 31.1. Preferred connexin antisense polynucleotides target connexin 43.

Conveniently, the oligodeoxynucleotide to connexin 43 is selected from: GTA ATT GCG GCA AGA AGA ATT GTT TCT GTC (SEQ.ID.NO:1); GTA ATT GCG GCA GGA GGA ATT GTT TCT GTC (SEQ.ID.NO:2); GGC AAG AGA CAC CAA AGA CAC TAC CAG CAT (SEQ.ID.NO:3), a polynucleotide having at least about 70 percent homology with SEQ.ID.NOS:1, 2, or 3 or a polynucleotide which hybridizes to connexin 43 mRNA under conditions of medium to high stringency.

In certain embodiments, the anti-connexin polynucleotide is effective to (a) prevent or retard keloid formation, (b) prevent or retard abnormal hypertrophic scar formation (c) prevent or retard excess scar formation and/or (d) inhibit intercellular communication by decreasing gap junction formation, in whole or in part. In certain embodiments, the anti-connexin polynucleotide is administered to skin tissue, or tissue ipened as a result of trauma or surgery. In one embodiment, the anti-connexin polynucleotide is administered topically. In other embodiments, the anti-connexin polynucleotide is implanted or instilled.

The invention further relates to an article of manufacture comprising: (a) a pharmaceutical composition having (i) an anti-connexin polynucleotide, and (ii) a pharmaceutically acceptable carrier, and (b) instructions for administering the pharmaceutical composition to a patient having, or at risk of having, an abnormal or excessive scar, including, for example, a keloid scar, a hypertrophic scar, an atrophic scar, a widespread scar, or other abnormal or excessive scarring. In certain embodiments, the instructions describe administration of the pharmaceutical composition to the patient to treat or prevent abnormal or excessive scar formation by excising an abnormal or excessive scar, for example, a keloid scar, a hypertrophic scar, an atrophic scar, a widespread scar, and administering the pharmaceutical composition in a quantity sufficient to prevent or reduce abnormal or excessive scarring at a site of the wound.

The invention also relates to a method of making an article of manufacture, which method comprises: combining (a) a container including a pharmaceutical composition comprising (i) an anti-connexin polynucleotide, and (ii) a pharmaceutically acceptable carrier, and (b) labeling instructions for treating a patient having an abnormal or excessive scar, including, for example, a keloid scar, a hypertrophic scar, an atrophic scar, or a widespread scar, by administering the pharmaceutical composition to a patient having an abnormal or excessive scar, including, for example, a keloid scar, a hypertrophic scar, an atrophic scar, or a widespread scar. In certain embodiments, the instructions describe administration of the pharmaceutical composition to the patient to treat an abnormal or excessive scar, including, for example, a keloid scar, a hypertrophic scar, an atrophic scar, a or a widespread scar, by excising the scar and administering the pharmaceutical composition in a quantity sufficient to prevent or abnormal or excessive scarring at a site of the wound.

The invention also relates to a method to determine the anti-hypertrophic scar activity of an anti-connexin polynucleotide to reduce abnormal or excessive scarring, comprising contacting cells or tissue at risk of developing an abnormal or excessive scar with an anti-connexin polynucleotide, and determining the anti-hypertropic scarring effect of said anti-connexin polynucleotide. In certain embodiments, the abnormal or excessive scar is a keloid scar, a hypertrophic scar, an atrophic scar, or a widespread scar. In one embodiment, the method to determine the anti-hypertrophic scar activity of an anti-connexin polynucleotide is carried out in vitro. In another embodiment, the method to determine the anti-hypertrophic scar activity of an anti-connexin polynucleotide is carried out in vivo.

The invention relates to a method to determine the anti-keloid activity of an anti-connexin polynucleotide, comprising contacting cells at risk of having a keloid with an anti-connexin polynucleotide, and determining the anti-keloid effect of said an anti-connexin polynucleotide. In one embodiment, the method to determine the anti-keloid activity of an anti-connexin polynucleotide method is carried out in vitro. In another embodiment, the method to determine the anti-keloid activity of an anti-connexin polynucleotide method is carried out in vivo.

Compositions and formulations of the invention useful in treating or preventing abnormal or excessive scarring (e.g. keloid or hypertrophic scarring) that employ anti-connexin polynucleotides, including connexin antisense polynucleotides, are described and claimed.

In one aspect, the invention provides a pharmaceutical composition useful in treating or preventing abnormal or excessive scarring (e.g. keloid or hypertrophic scarring) comprising one or more anti-connexin polynucleotides (e.g. connexin antisense polynucleotides). Preferably, the pharmaceutical composition further comprises a pharmaceutically acceptable carrier, diluent or excipient. For example, the inventions include pharmaceutical compositions comprising (a) a therapeutically effect amount of a pharmaceutically acceptable connexin antisense polynucleotide and (b) a pharmaceutically acceptable carrier or diluent.

The invention also includes pharmaceutical compositions useful in treating or preventing abnormal or excessive scarring (e.g. keloid or hypertrophic scarring) comprising (a) a therapeutically effective amount of an anti-connexin polynucleotide, and (b) a therapeutically effective amount of one or more therapeutic agents. The invention includes pharmaceutical compositions useful in treating or preventing abnormal or excessive scarring (e.g. keloid or hypertrophic scarring) comprising (a) a therapeutically effective amount of an anti-connexin polynucleotide, and (b) a therapeutically effective amount of one or more agents useful in wound healing. The invention includes pharmaceutical compositions useful in treating or preventing abnormal or excessive scarring (e.g. keloid or hypertrophic scarring) comprising (a) a therapeutically effective amount of an anti-connexin polynucleotide, and (b) a therapeutically effective amount of one or more protein synthesis inhibitors. Preferably, the pharmaceutical compositions further comprise a pharmaceutically acceptable carrier, diluent or excipient.

Pharmaceutical compositions useful in treating or preventing abnormal or excessive scarring (e.g. keloid or hypertrophic scarring) are provided for combined, simultaneous, separate sequential or sustained administration. In one embodiment, a composition comprising one or more anti-connexin polynucleotides is administered at or about the same time as one or more therapeutic agents, agents useful for wound healing and/or protein synthesis inhibitors.

Pharmaceutical compositions useful in treating or preventing abnormal or excessive scarring (e.g., keloid or hypertrophic scarring) are also provided in the form of a combined preparation, for example, as an admixture of one or more anti-connexin polynucleotides and one or more other agents useful for wound healing, e.g., growth factors that are effective in promoting or improving wound healing, such as platelet derived growth factor, epidermal growth factor, fibroblast growth factor (e.g., FGF2), vascular endothelial growth factor, and transforming growth factor β3, and/or cytokines that are effective in promoting or improving wound healing, such as IL-7 and IL-10, and/or other agents that are effective in promoting or improving wound healing, such as IGF (e.g., IGF-1) and IGFBP (e.g., IGFBP-2).

The term “a combined preparation” includes a “kit of parts” in the sense that the combination partners as defined above can be dosed independently or by use of different fixed combinations with distinguished amounts of the combination partners (a) and (b), i.e. simultaneously, separately or sequentially. The parts of the kit can then, for example, be administered simultaneously or chronologically staggered, that is at different time points and with equal or different time intervals for any part of the kit of parts.

In a preferred embodiment, the administration of a combined preparation will have fewer administration time points and/or increased time intervals between administrations as a result of such combined use.

In another aspect, the invention includes methods for administering a therapeutically effective amount of one or more pharmaceutically acceptable connexin antisense polynucleotides formulated in a delayed release preparation, a slow release preparation, an extended release preparation, a controlled release preparation, and/or in a repeat action preparation to a subject to treat and/or prevent abnormal or excessive scarring.

In a further aspect, the invention includes transdermal patches, dressings, pads, wraps, matrices and bandages capable of being adhered or otherwise associated with the skin of a subject, said articles being capable of delivering a therapeutically effective amount of one or more pharmaceutically acceptable anti-connexin polynucleotides, e.g., connexin antisense polynucleotides to a subject to treat or prevent abnormal or excessive scarring.

The invention includes devices useful in treating or preventing abnormal or excessive scarring (e.g. keloid or hypertrophic scarring) containing therapeutically effective amounts of one or more pharmaceutically acceptable anti-connexin polynucleotides, e.g., connexin antisense polynucleotides, for example, a rate-controlling membrane enclosing a drug reservoir and a monolithic matrix device. These devices may be employed for the treatment of subjects in need thereof as disclosed herein. Suitably the wound dressing or matrix is provided including the form of a solid substrate with an anti-connexin polynucleotide, e.g., a connexin antisense polynucleotide, either alone or in combination with one or more therapeutic agents, agents useful for wound healing, and/or protein synthesis inhibitors, dispersed on or in the solid substrate. In one embodiment the pharmaceutical product of the invention is provided in combination with a wound dressing or wound healing promoting matrix. Preferred anti-connexin polynucleotides and connexin antisense polynucleotides are anti-connexin 43 polynucleotides and connexin 43 antisense polynucleotides.

The invention also relates to an article of manufacture useful in treating or preventing abnormal or excessive scarring (e.g. keloid or hypertrophic scarring or other abnormal or excessive scarring) comprising: (a) a pharmaceutical composition having (i) a therapeutically effective amount of an anti-connexin polynucleotide, and (ii) a pharmaceutically acceptable carrier, and (b) instructions for administering the pharmaceutical composition to a subject having or at risk for having an abnormal or excessive scar, e.g., a keloid scar, a hypertrophic scar, or other abnormal or excessive scar. In certain embodiments, the instructions describe administration of the pharmaceutical composition to the subject to treat a keloid scar, a hypertrophic scar, or other abnormal or excessive scar by excising the scar and administering the pharmaceutical composition in a quantity sufficient to prevent or reduce abnormal or excessive scarring at a site of the wound. Preferred anti-connexin polynucleotides and connexin antisense polynucleotides are anti-connexin 43 polynucleotides and connexin 43 antisense polynucleotides. In one embodiment, the composition further comprises a second composition comprising a therapeutically effective amount of one or more therapeutic agents, agents useful for wound healing and/or protein synthesis inhibitors. In one embodiment, the article of manufacture further comprises a second composition comprising a therapeutically effective amount of one or more therapeutic agents, agents useful for wound healing, and/or protein synthesis inhibitors.

The invention relates to a method of making an article of manufacture useful in treating or preventing abnormal or excessive scarring (e.g. keloid or hypertrophic scarring or other abnormal or excessive scarring), which method comprises: combining (a) a container including a pharmaceutical composition comprising (i) a therapeutically effective amount of an anti-connexin polynucleotide, and (ii) a pharmaceutically acceptable carrier, and (b) labeling and/or other instructions for treating a subject having or at risk for having a keloid or other abnormal or excessive scar by administering the pharmaceutical composition to a subject. In certain embodiments, the instructions describe administration of the pharmaceutical composition to the subject to treat a keloid scar, a hypertrophic scar, or other abnormal or excessive scar by excising the scar and administering the pharmaceutical composition in a quantity sufficient to prevent or reduce abnormal or excessive scarring at a site of the wound. Preferred anti-connexin polynucleotides and connexin antisense polynucleotides are anti-connexin 43 polynucleotides and connexin 43 antisense polynucleotides; In one embodiment, the composition further comprises a second composition comprising a therapeutically effective amount of one or more therapeutic agents, agents useful for wound healing and/or protein synthesis inhibitors. In one embodiment, the article of manufacture further comprises a second composition comprising a therapeutically effective amount of one or more therapeutic agents, agents useful for wound healing, and/or protein synthesis inhibitors.

### DETAILED DESCRIPTION OF THE INVENTION

### Definitions

As used herein, “subject” refers to any mammals, including humans, domestic and farm animals, and zoo, sports, or pet animals, such as dogs, horses, cats, sheep, pigs, cows, etc. The preferred mammal herein is a human, including adults, children, and the elderly.

As used herein, “preventing” means preventing in whole or in part, ameliorating or controlling, or reducing, lessening, decreasing or retarding.

As used herein, a “therapeutically effective amount” or “effective amount” in reference to the compounds or compositions of the instant invention refers to the amount sufficient to induce a desired biological, pharmaceutical, or therapeutic result. That result can be alleviation of the signs, symptoms, or causes of a disease or disorder or condition, or any other desired alteration of a biological system. In the present invention, the result will involve the prevention and/or reduction of abnormal or excessive scarring, as well as prevention and/or reduction of excessive scar formation and other types of abnormal or excessive proliferation of tissue, including keloid scars, hypertrophic scars, widespread scars, and atrophic scars.

As used herein, the term “treating” refers to both therapeutic treatment and prophylactic or preventative measures. Those in need of treatment include those already with the disorder as well as those prone to having the disorder or diagnosed with the disorder or those in which the disorder is to be prevented.

As used herein, “simultaneously” is used to mean that the one or more anti-connexin polynucleotides,alone or in combination with one or more therapeutic agents, agents useful for wound healing and/or protein synthesis inhibitors are administered concurrently, whereas the term “in combination” is used to mean the polynucleotides and/or agents are administered, if not simultaneously or in physical combination, then “sequentially” within a timeframe that they both are available to act therapeutically. Thus, administration “sequentially” may permit one polynucleotide or agent to be administered within minutes (for example, 1, 2, 3, 4, 5, 10, 15, 20, 25, 30) minutes or a matter of hours, days, weeks or months after the other polynucleotide or agent provided that both are concurrently present in therapeutically effective amounts. The time delay between administration of the components will vary depending on the exact nature of the components, the interaction there between, and their respective half-lives.

As used herein, an “anti-connexin polynucleotide” or “anti-connexin agent” decreases or inhibits expression of connexin mRNA and/or protein. Anti-connexin polynucleotides include, without limitation, antisense compounds such as antisense polynucleotides, other polynucleotides (such as polynucleotides having siRNA or ribozyme functions). Suitable examples of an anti-connexin polynucleotide include an antisense polynucleotide to a connexin. Accordingly, suitable anti-connexin polynucleotides include, for example, antisense polynucleotides (e.g., connexin 43 antisense polynucleotides) that modulate expression or activity of connexins and gap junctions in selected tissues, cells, and subjects. Exemplary anti-connexin polynucleotides are further described herein.

### Anti-Connexin Polynucleotides

Anti-connexin polynucleotides include connexin antisense polynucleotides as well as polynucleotides which have functionalities which enable them to downregulate or inhibit connexin expression (for example, by downregulation or inhibition of mRNA transcription or translation). In the case of downregulation, this will have the effect of reducing direct cell-cell communication by gap junctions at the site at which connexin expression is downregulated.

Suitable anti-connexin polynucleotides include RNAi polynucleotides and siRNA polynucleotides.

Synthesis of antisense polynucleotides and other anti-connexin polynucleotides such as RNAi, siRNA, and ribozyme polynucleotides as well as polynucleotides having modified and mixed backbones is known to those of skill in the art. See e.g. Stein C. A. and Krieg A. M. (eds), Applied Antisense Oligonucleotide Technology, 1998 (Wiley-Liss).

According to one aspect, the downregulation or inhibition of connexin expression may be based generally upon the antisense approach using antisense polynucleotides (such as DNA or RNA polynucleotides), and more particularly upon the use of antisense oligodeoxynucleotides (ODN). These polynucleotides (e.g., ODN) target the connexin protein (s) to be downregulated. Typically the polynucleotides are single stranded, but may be double stranded.

The antisense polynucleotide may inhibit transcription and/or translation of a connexin. Preferably the polynucleotide is a specific inhibitor of transcription and/or translation from the connexin gene or mRNA, and does not inhibit transcription and/or translation from other genes or mRNAs. The product may bind to the connexin gene or mRNA either (i) 5′ to the coding sequence, and/or (ii) to the coding sequence, and/or (iii) 3′ to the coding sequence.

The antisense polynucleotide is generally antisense to a connexin mRNA. Such a polynucleotide may be capable of hybridizing to the connexin mRNA and may thus inhibit the expression of connexin by interfering with one or more aspects of connexin mRNA metabolism including transcription, mRNA processing, mRNA transport from the nucleus, translation or mRNA degradation. The antisense polynucleotide typically hybridizes to the connexin mRNA to form a duplex which can cause direct inhibition of translation and/or destabilization of the mRNA. Such a duplex may be susceptible to degradation by nucleases.

The antisense polynucleotide may hybridize to all or part of the connexin mRNA. Typically the antisense polynucleotide hybridizes to the ribosome binding region or the coding region of the connexin mRNA. The polynucleotide may be complementary to all of or a region of the connexin mRNA. For example, the polynucleotide may be the exact complement of all or a part of connexin mRNA. However, absolute complementarity is not required and polynucleotides which have sufficient complementarity to form a duplex having a melting temperature of greater than about 20° C., 30° C. or 40° C. under physiological conditions are particularly suitable for use in the present invention.

Thus the polynucleotide is typically a homologue of a sequence complementary to the mRNA. The polynucleotide may be a polynucleotide which hybridizes to the connexin mRNA under conditions of medium to high stringency such as 0.03M sodium chloride and 0.03M sodium citrate at from about 50° C. to about 60° C.

For certain aspects, suitable polynucleotides are typically from about 6 to 40 nucleotides in length. Preferably a polynucleotide may be from about 12 to about 35 nucleotides in length, or alternatively from about 12 to about 20 nucleotides in length or more preferably from about 18 to about 32 nucleotides in length. According to an alternative aspect, the polynucleotide may be at least about 40, for example at least about 60 or at least about 80, nucleotides in length and up to about 100, about 200, about 300, about 400, about 500, about 1000, about 2000 or about 3000 or more nucleotides in length.

The connexin protein or proteins targeted by the polynucleotide will be dependent upon the site at which downregulation is to be effected. This reflects the non-uniform make-up of gap junction(s) at different sites throughout the body in terms of connexin sub-unit composition. The connexin is a connexin that naturally occurs in a human or animal in one aspect or naturally occurs in the tissue in which connexin expression or activity is to be decreased. The connexin gene (including coding sequence) generally has homology with the coding sequence of one or more of the specific connexins mentioned herein, such as homology with the connexin 43 coding sequence shown in Table 2. The connexin is typically an α or β connexin. Preferably the connexin is an α connexin and is expressed in the tissue to be treated.

Some connexin proteins are however more ubiquitous than others in terms of distribution in tissue. One of the most widespread is connexin 43. Polynucleotides targeted to connexin 43 are particularly suitable for use in the present invention. In other aspects other connexins are targeted.

Anti-connexin polynucleotides include connexin antisense polynucleotides as well as polynucleotides which have functionalities which enable them to downregulate connexin expression. Other suitable anti-connexin polynucleotides include RNAi polynucleotides and siRNA polynucleotides.

In one preferred aspect, the antisense polynucleotides are targeted to the mRNA of one connexin protein only. Most preferably, this connexin protein is connexin 43. In another aspect, connexin protein is connexin 26, 30, 31.1, 32, 36, 37, 40, or 45. In other aspects, the connexin protein is connexin 30.3, 31, 40.1, or 46.6.

It is also contemplated that polynucleotides targeted to separate connexin proteins be used in combination (for example 1, 2, 3, 4 or more different connexins may be targeted). For example, polynucleotides targeted to connexin 43, and one or more other members of the connexin family (such as connexin 26, 30, 30.3, 31.1, 32, 36, 37, 40, 40.1, 45, and 46.6) can be used in combination.

Alternatively, the antisense polynucleotides may be part of compositions which may comprise polynucleotides to more than one connexin protein. Preferably, one of the connexin proteins to which polynucleotides are directed is connexin 43. Other connexin proteins to which oligodeoxynucleotides are directed may include, for example, connexins 26, 30, 30.3, 31.1, 32, 36, 37, 40, 40.1, 45, and 46.6. Suitable exemplary polynucleotides (and ODNs) directed to various connexins are set forth in Table 1.

Individual antisense polynucleotides may be specific to a particular connexin, or may target 1, 2, 3 or more different connexins. Specific polynucleotides will generally target sequences in the connexin gene or mRNA which are not conserved between connexins, whereas non-specific polynucleotides will target conserved sequences for various connexins.

The polynucleotides for use in the invention may suitably be unmodified phosphodiester oligomers. Such oligodeoxynucleotides may vary in length. A 30 mer polynucleotide has been found to be particularly suitable.

Many aspects of the invention are described with reference to oligodeoxynucleotides. However it is understood that other suitable polynucleotides (such as RNA polynucleotides) may be used in these aspects.

The antisense polynucleotides may be chemically modified. This may enhance their resistance to nucleases and may enhance their ability to enter cells. For example, phosphorothioate oligonucleotides may be used. Other deoxynucleotide analogs include methylphosphonates, phosphoramidates, phosphorodithioates, N3′P5′-phosphoramidates and oligoribonucleotide phosphorothioates and their 2′-O-alkyl analogs and 2′-O-methylribonucleotide methylphosphonates. Alternatively mixed backbone oligonucleotides (“MBOs”) may be used. MBOs contain segments of phosphothioate oligodeoxynucleotides and appropriately placed segments of modified oligodeoxy- or oligoribonucleotides. MBOs have segments of phosphorothioate linkages and other segments of other modified oligonucleotides, such as methylphosphonate, which is non-ionic, and very resistant to nucleases or 2′-O-alkyloligoribonucleotides. Methods of preparing modified backbone and mixed backbone oligonucleotides are known in the art.

The precise sequence of the antisense polynucleotide used in the invention will depend upon the target connexin protein. In one embodiment, suitable connexin antisense polynucleotides can include polynucleotides such as oligodeoxynucleotides selected from the following sequences set forth in Table 1:

TABLE 1

| | | |
|---|---|---|
| 5′ GTA ATT GCG GCA AGA AGA ATT GTT TCT GTC 3′ | (connexin 43) | (SEQ.ID.NO: 1) |
| 5′ GTA ATT GCG GCA GGA GGA ATT GTT TCT GTC 3′ | (connexin 43) | (SEQ.ID.NO: 2) |
| 5′ GGC AAG AGA CAC CAA AGA CAC TAC CAG CAT 3′ | (connexin 43) | (SEQ.ID.NO: 3) |
| 5′ TCC TGA GCA ATA CCT AAC GAA CAA ATA 3′ | (connexin 26) | (SEQ.ID.NO: 4) |
| 5′ CAT CTC CTT GGT GCT CAA CC 3′ | (connexin 37) | (SEQ.ID.N0: 5) |
| 5′ CTG AAG TCG ACT TGG CTT GG 3′ | (connexin 37) | (SEQ.ID.N0: 6) |
| 5′ CTC AGA TAG TGG CCA GAA TGC 3′ | (connexin 30) | (SEQ.ID.NO: 7) |
| 5′ TTG TCC AGG TGA CTC CAA GG 3′ | (connexin 30) | (SEQ.ID.NO: 8) |
| 5′ CGT CCG AGC CCA GAA AGA TGA GGT C 3′ | (connexin 31.1) | (SEQ.ID.NO: 9) |
| 5′ AGA GGC GCA CGT GAG ACA C 3′ | (connexin 31.1) | (SEQ.ID.NO: 10) |
| 5′ TGA AGA CAA TGA AGA TGT T 3′ | (connexin 31.1) | (SEQ.ID.N0: 11) |
| 5′ TTT CTT TTC TAT GTG CTG TTG GTG A 3′ | (connexin 32) | (SEQ.ID.NO: 12) |

Suitable polynucleotides for the preparation of the combined polynucleotide compositions described herein include for example, polynucleotides to connexin 43 and polynucleotides for connexins 26, 30, 31.1, 32 and 37 as described in Table 1 above.

Although the precise sequence of the antisense polynucleotide used in the invention will depend upon the target connexin protein, for connexin 43, antisense polynucleotides having the following sequences have been found to be particularly suitable:

(SEQ.ID.NO: 1)
GTA ATT GCG GCA AGA AGA ATT GTT TCT GTC;
(SEQ.ID.NO: 2)
GTA ATT GCG GCA GGA GGA ATT GTT TCT GTC;
and
(SEQ.ID.NO: 3)
GGC AAG AGA CAC CAA AGA CAC TAC CAG CAT.

For example, suitable antisense polynucleotides for connexins 26, 31.1 and 32 have the following sequences:

(SEQ.ID.NO: 4)
5′ TCC TGA GCA ATA CCT AAC GAA CAA ATA;
(connexin 26)
(SEQ.ID.NO: 9)
5′ CGT CCG AGC CCA GAA AGA TGA GGT C;
(connexin 31.1)
and
(SEQ.ID.NO: 12)
5′ TTT CTT TTC TAT GTG CTG TTG GTG A.
(connexin 32)

Other connexin antisense polynucleotide sequences useful according to the methods of the present invention include:

(SEQ.ID.NO: 5)
5′ CAT CTC CTT GGT GCT CAA CC 3′; (connexin 37)
(SEQ.ID.NO: 6)
5′ CTG AAG TCG ACT TGG CTT GG 3′; (connexin 37)
(SEQ.ID.NO: 7)
5′ CTC AGA TAG TGG CCA GAA TGC 3′; (connexin 30)
(SEQ.ID.NO: 8)
5′ TTG TCC AGG TGA CTC CAA GG 3′; (connexin 30)
(SEQ.ID.NO: 10)
5′ AGA GGC GCA CGT GAG ACA C 3′; (connexin 31.1)
and
(SEQ.ID.NO: 11)
5′ TGA AGA CAA TGA AGA TGT T 3′. (connexin 31.1)

Polynucleotides, including ODN's, directed to connexin proteins can be selected in terms of their nucleotide sequence by any convenient, and conventional, approach. For example, the computer programs MacVector and OligoTech (from Oligos etc. Eugene, Oreg., USA) can be used. Once selected, the ODN's can be synthesized using a DNA synthesizer.

### Polynucleotide Homologues

Anti-connexin polynucleotides also include polynucleotide homologues. Homology and homologues are discussed herein (for example, the polynucleotide may be a homologue of a complement to a sequence in connexin mRNA). Such a polynucleotide typically has at least about 70% homology, preferably at least about 80%, at least about 90%, at least about 95%, at least about 97% or at least about 99% homology with the relevant sequence, for example over a region of at least about 15, at least about 20, at least about 40, at least about 100 more contiguous nucleotides (of the homologous sequence).

Homology may be calculated based on any method in the art. For example the UWGCG Package provides the BESTFIT program, which can be used to calculate homology (for example used on its default settings) (Devereux et al. (1984) Nucleic Acids Research 12, p387-395). The PILEUP and BLAST algorithms can be used to calculate homology or line up sequences (typically on their default settings), for example as described in Altschul S. F. (1993) J Mol Evol 36: 290-300; Altschul, S, F et al (1990) J Mol Biol 215: 403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached.

The BLAST algorithm parameters W, T and X determine the sensitivity and speed of the alignment. The BLAST program uses as defaults a word length (W), the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to a second sequence is less than about 1, preferably less than about 0.1, more preferably less than about 0.01, and most preferably less than about 0.001.

The homologous sequence typically differs from the relevant sequence by at least about (or by no more than about) 2, 5, 10, 15, 20 more mutations (which may be substitutions, deletions or insertions). These mutations may be measured across any of the regions mentioned above in relation to calculating homology.

The homologous sequence typically hybridizes selectively to the original sequence at a level significantly above background. Selective hybridization is typically achieved using conditions of medium to high stringency (for example 0.03M sodium chloride and 0.03M sodium citrate at from about 50° C. to about 60° C.). However, such hybridization may be carried out under any suitable conditions known in the art (see Sambrook et al. (1989), Molecular Cloning: A Laboratory Manual). For example, if high stringency is required, suitable conditions include 0.2×SSC at 60° C. If lower stringency is required, suitable conditions include 2×SSC at 60° C.

### Therapeutic Agents

Therapeutic agents include pharmaceutically acceptable agents useful in the treatment of wounds or the promotion of wound-healing, whether currently existing and known or later developed. Therapeutic agents include, for example, anti-infectives, anesthetics, analgesics, antibiotics, narcotics, and steroidal and non-steroidal anti-inflammatory agents. Preferred therapeutic agents include topical steroid anti-inflammatory agents, antimicrobial agents, local and topical anesthetics, and topical opioids. In certain embodiments, one, two three, four, five or six therapeutic agents may be used in combination. The therapeutic agents are not an anti-connexin peptide, an anti-connexin peptidomimetic, a gap junction or hemichannel phosphorylation compound (e.g., a gap junction or hemichannel phosphorylation compound that closes a gap junction or hemichannel), or a connexin carboxy-terminal peptide (e.g., a connexin carboxy-terminal peptide that blocks or otherwise inhibits interaction with a ZO-1 protein).

### Protein Synthesis Inhibitors

Protein synthesis inhibitors include pharmaceutically acceptable agents useful in the preventing or treating scar formations. Protein synthesis inhibitors include steroids, including but not limited to corticosteroids and glucocorticosteroids, such as triamcinolone acetonide (also known as KENALOG™), and Vitamin E (α-tocopherol) (Ehrlich et al. 1972, Ann. Surg. 75:235).

### Agents Useful for Wound Healing

As used herein, agents useful for wound healing include stimulators, enhancers or positive mediators of the wound healing cascade which 1) promote or accelerate the natural wound healing process or 2) reduce effects associated with improper wound healing, which effects include, for example, adverse inflammation, epithelialization, angiogenesis and matrix deposition, and excess scarring.

Agents useful for wound healing are not an anti-connexin peptide, an anti-connexin peptidomimetic, a gap junction or hemichannel phosphorylation compound (e.g., a gap junction or hemichannel phosphorylation compound that closes a gap junction or hemichannel), or a connexin carboxy-terminal peptide (e.g., a connexin carboxy-terminal peptide that blocks or otherwise inhibits interaction with a ZO-1 protein).

Positive mediators, enhancers and stimulators include for example, an agent which may stimulate, enhance, facilitate, or accelerate (i.e., agonize) the quantity, quality or efficacy of wound healing or the active wound healing process, or a wound healing-associated growth factor or cytokine at a wound site, or the activation of a wound healing-associated growth factor or cytokine receptor. Such agents may include a wound healing-associated growth factor or cytokine or a partially modified form of a wound healing-associated growth factor or cytokine, for example. A partially modified form of wound healing-associated growth factor or cytokine may, for example, have a longer half-life than the natural wound healing-associated growth factor or cytokine. Alternatively, it may be an inhibitor of wound healing-associated growth factor or cytokine metabolism.

Partial modification of such an agent may be by way of addition, deletion or substitution of amino acid residues. A substitution may for example be a conserved substitution. Hence a partially modified molecule may be a homologue of the molecule from which it was derived. It may have at least about 40%, for example about 50, 60, 70, 80, 90 or 95%, homology with the molecule from which it is derived.

As used herein, agents useful for wound healing may include for example, wound-healing-promoting or scar-reducing agents for wound treatment modalities now known in the art or later-developed; exemplary factors, agents or modalities including natural or synthetic growth factors, cytokines, or modulators thereof to promote wound healing, wound healing promoting bioengineered matrix, dressings bandages, and the like. Suitable examples may include, but not limited to 1) topical or dressing and related therapies and debriding agents (such as, for example, Santyl® collagenase) and Iodosorb® (cadexomer iodine); 2) antimicrobial agents, including systemic or topical creams or gels, including, for example, silver-containing agents such as SAGs (silver antimicrobial gels), (CollaGUARD™, Innocoll, Inc) (purified type-I collagen protein based dressing), CollaGUARD Ag (a collagen-based bioactive dressing impregnated with silver for infected wounds or wounds at risk of infection), DermaSIL™ (a collagen-synthetic foam composite dressing for deep and heavily exuding wounds); 3) cell therapy or bioengineered skin, skin substitutes, and skin equivalents, including, for example, Dermograft (3-dimensional matrix cultivation of human fibroblasts that secrete cytokines and growth factors), Apligraf® (human keratinocytes and fibroblasts), Graftskin® (bilayer of epidermal cells and fibroblasts that is histologically similar to normal skin and produces growth factors similar to those produced by normal skin), TransCyte (a Human Fibroblast Derived Temporary Skin Substitute) and Oasis® (an active biomaterial that comprises both growth factors and extracellular matrix components such as collagen, proteoglycans, and glycosaminoglycans); 4) cytokines, growth factors or hormones (both natural and synthetic) introduced to the wound to promote wound healing, including, for example, NGF, NT3, BDGF, integrins, plasmin, semaphoring, blood-derived growth factor, keratinocyte growth factor, tissue growth factor, TGF-alpha, TGF-beta, PDGF (one or more of the three subtypes may be used: AA, AB, and B), PDGF-BB, TGF-beta 3, factors that modulate the relative levels of TGFβ3, TGFβ1, and TGFβ2 (e.g., Mannose-6-phosphate), sex steroids, including for example, estrogen, estradiol, or an oestrogen receptor agonist selected from the group consisting of ethinyloestradiol, dienoestrol, mestranol, oestradiol, oestriol, a conjugated oestrogen, piperazine oestrone sulphate, stilboestrol, fosfesterol tetrasodium, polyestradiol phosphate, tibolone, a phytoestrogen, 17-beta-estradiol; thymic hormones such as Thymosin-beta-4, EGF, HB-EGF, fibroblast growth factors (e.g., FGF1, FGF2, FGF7), keratinocyte growth factor, TNF, interleukins family of inflammatory response modulators such as, for example, IL-10, IL-1, IL-2, IL-6, IL-8, and IL-10 and modulators thereof; INFs (INF-alpha, -beta, and -delta); stimulators of activin or inhibin, and inhibitors of interferon gamma prostaglandin E2 (PGE2) and of mediators of the adenosine 3′,5′-cyclic monophosphate (cAMP) pathway; adenosine A1 agonist, adenosine A2 agonist or 5) other agents useful for wound healing, including, for example, both natural or synthetic homologues, agonist and antagonist of VEGF, VEGFA, IGF; IGF1, proinflammatory cytokines, GM-CSF, and leptins and 6) IGF-1 and KGF cDNA, autologous platelet gel, hypochlorous acid (Sterilox® lipoic acid, nitric oxide synthase3, matrix metalloproteinase 9 (MMP-9), CCT-ETA, alphavbeta6 integrin, growth factor-primed fibroblasts and Decorin, silver containing wound dressings, Xenaderm™, papain wound debriding agents, lactoferrin, substance P, collagen, and silver-ORC, placental alkaline phosphatase or placental growth factor, modulators of hedgehog signaling, modulators of cholesterol synthesis pathway, and APC (Activated Protein C), keratinocyte growth factor, TNF, Thromboxane A2, NGF, BMP bone morphogenetic protein, CTGF (connective tissue growth factor), wound healing chemokines, decorin, modulators of lactate induced neovascularization, cod liver oil, placental alkaline phosphatase or placental growth factor, and thymosin beta 4. In certain embodiments, one, two three, four, five or six agents useful for wound healing may be used in combination.

It is to be understood that the agents useful for wound healing (including for example, growth factors and cytokines) above encompass all naturally occurring polymorphs (for example, polymorphs of the growth factors or cytokines). Also, functional fragments, chimeric proteins comprising one of said agents useful for wound healing or a functional fragment thereof, homologues obtained by analogous substitution of one or more amino acids of the agent useful for wound healing, and species homologues are encompassed. It is contemplated that one or more agents useful for wound healing may be a product of recombinant DNA technology, and one or more agents useful for wound healing may be a product of transgenic technology. For example, platelet derived growth factor may be provided in the form of a recombinant PDGF or a gene therapy vector comprising a coding sequence for PDGF.

A fragment or partially modified form thereof refers to a fragment or partially modified form of the agent useful for wound healing which retains the biological or wound healing functionality of the factor, although it may of course have additional functionality. Partial modification may, for example, be by way of addition, deletion or substitution of amino acid residues. For example, a substitution may be a conserved substitution. Hence the partially modified molecules may be homologues of the agent useful for wound healing. They may, for example, have at least about 40% homology with said factor. They may for example have at least about 50, 60, 70, 80, 90 or 95% homology with said factor. For example, in certain embodiments, IL-10 or a fragment or a partially modified form thereof may be administered at a concentration of between about 1 μM and about 10 μM. It may be administered at a concentration of between about 2.5 μM and about 5 μM. In certain other embodiments, IL-10 or a fragment or a partially modified form thereof may be administered immediately prior to wound healing, but may be effective if administered within about 7 days of wounding. It could be administered on at least two occasions.

### Dosage Forms and Formulations and Administration

The anti-connexin polynucleotides of the invention (typically in the form of the formulations discussed herein) may be administered to a subject in need of treatment, such as a subject with (or at risk for having) an abnormal or excessive scar, including any of the abnormal or excessive scars mentioned herein. The condition of the subject can thus be improved. The anti-connexin polynucleotide may be used in the treatment of the subject's body by therapy. They may be used in the manufacture of a medicament to treat or prevent any abnormal or excessive scar, including any of the abnormal or excessive scars mentioned herein.

Thus, in accordance with the invention, there are provided formulations by which cell-cell communication can be downregulated in a transient and site-specific manner for the treatment and/or prevention of abnormal or excessive scarring.

The anti-connexin polynucleotide may be conveniently formulated with a pharmaceutically acceptable carrier to give the desired final concentration.

The anti-connexin polynucleotide may be present in a substantially isolated form. It will be understood that the product may be mixed with carriers or diluents which will not interfere with the intended purpose of the product and still be regarded as substantially isolated. A product of the invention may also be in a substantially purified form, in which case it will generally comprise at least about 80%, 85%, or 90%, including, for example, at least about 95%, at least about 98% or at least about 99% of the polynucleotide or dry mass of the preparation.

Depending on the intended route of administration, the pharmaceutical products, pharmaceutical compositions, combined preparations and medicaments of the invention may, for example, take the form of solutions, suspensions, instillations, sprays, salves, creams, wound dressings, gels, foams, ointments, emulsions, lotions, paints, sustained release formulations, or powders, and typically contain about 1 to 95%, 0.01% to about 1% of active ingredient(s), about 1%-50% or active ingredient(s), about 2%-60% of active ingredient(s), about 2%-70% of active ingredient(s), or up to about 90% of active ingredient(s). Other suitable formulations include pluronic gel-based formulations, carboxymethylcellulose(CMC)-based formulations, and hyroxypropylmethylcellulose(HPMC)-based formulations. Other useful formulations include slow or delayed release preparations.

Gels or jellies may be produced using a suitable gelling agent including, but not limited to, gelatin, tragacanth, or a cellulose derivative and may include glycerol as a humectant, emollient, and preservative. Ointments are semi-solid preparations that consist of the active ingredient incorporated into a fatty, waxy, or synthetic base. Examples of suitable creams include, but are not limited to, water-in-oil and oil-in-water emulsions. Water-in-oil creams may be formulated by using a suitable emulsifying agent with properties similar, but not limited, to those of the fatty alcohols such as cetyl alcohol or cetostearyl alcohol and to emulsifying wax. Oil-in-water creams may be formulated using an emulsifying agent such as cetomacrogol emulsifying wax. Suitable properties include the ability to modify the viscosity of the emulsion and both physical and chemical stability over a wide range of pH. The water soluble or miscible cream base may contain a preservative system and may also be buffered to maintain an acceptable physiological pH.

Foam preparations may be formulated to be delivered from a pressurized aerosol canister, via a suitable applicator, using inert propellants. Suitable excipients for the formulation of the foam base include, but are not limited to, propylene glycol, emulsifying wax, cetyl alcohol, and glyceryl stearate. Potential preservatives include methylparaben and propylparaben.

The anti-connexin polynucleotide may be mixed with physiological tolerable and compatible diluents, excipients and preferably the polynucleotides of the invention are combined with a pharmaceutically acceptable carrier or diluent to produce a pharmaceutical composition. Suitable carriers and diluents include isotonic saline solutions, for example phosphate-buffered saline. Suitable diluents and excipients also include, for example, water, saline, dextrose, glycerol, or the like, and combinations thereof. In addition, if desired substances such as wetting or emulsifying agents, stabilizing or ph buffering agents may also be present.

The term “pharmaceutically acceptable carrier” refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, and amino acid copolymers.

Pharmaceutically acceptable salts can also be present, e.g., mineral acid salts such as hydrochlorides, hydrobromides, phosphates, sulfates, and the like; and the salts of organic acids such as acetates, propionates, malonates, benzoates, and the like.

Suitable carrier materials include any carrier or vehicle commonly used as a base for creams, lotions, sprays, foams, gels, emulsions, lotions or paints for topical administration. Examples include emulsifying agents, inert carriers including hydrocarbon bases, emulsifying bases, non-toxic solvents or water-soluble bases. Particularly suitable examples include pluronics, HPMC, CMC and other cellulose-based ingredients, lanolin, hard paraffin, liquid paraffin, soft yellow paraffin or soft white paraffin, white beeswax, yellow beeswax, cetostearyl alcohol, cetyl alcohol, dimethicones, emulsifying waxes, isopropyl myristate, microcrystalline wax, oleyl alcohol and stearyl alcohol.

Preferably, the pharmaceutically acceptable carrier or vehicle is a gel, suitably a nonionic polyoxyethylene-polyoxypropylene copolymer gel, for example, a Pluronic gel, preferably Pluronic F-127 (BASF Corp.). This gel is particularly preferred as it is a liquid at low temperatures but rapidly sets at physiological temperatures, which confines the release of the OND component to the site of application or immediately adjacent that site.

An auxiliary agent such as casein, gelatin, albumin, glue, sodium alginate, carboxymethylcellulose, methylcellulose, hydroxyethylcellulose or polyvinyl alcohol may also be included in the formulation of the invention.

The pharmaceutical composition may be formulated to provide sustained release of the anti-connexin polynucleotide alone or in combination with one or more wound modulating agents.

The one or more anti-connexin polynucleotides may be administered by the same or different routes. Preferably said one or more anti-connexin polynucleotides are delivered by topical administration (peripherally or directly to a site), including but not limited to topical administration using solid supports (such as dressings and other matrices) and medicinal formulations (such as gels, mixtures, suspensions and ointments). In one embodiment, the solid support comprises a biocompatible membrane. In another embodiment, the solid support comprises a dressing or matrix. In one embodiment a wash solution comprising the one or more anti-connexin polynucleotides can be used locally to prevent or decrease excessive scarring including keloids, hypertrophic scars, atrophic scars, and widespread scars.

The anti-connexin agents, including for example the anti-connexin polynucleotides of the invention, may also be delivered over an extended period of time. While the delivery period will be dependent upon both the site at which the downregulation is to be induced and the therapeutic effect which is desired, continuous or slow-release delivery for about 1-2 hours, about 2-4 hours, about 4-6 hours, about 6-8, or about 24 hours or longer is provided. In accordance with the present invention, this is achieved by inclusion of the polynucleotides in a formulation together with a pharmaceutically acceptable carrier or vehicle, particularly in the form of a formulation for continuous or slow-release administration.

As noted, the one or more anti-connexin polynucleotides may be administered before, during, immediately following surgery or wounding, for example, preferably within about 24, about 12, about 10, about 9, about 8, about 7, about 6, about 5, about 4, about 3, about 2 hours or within about 60, about 45, about 30, about 15, about 10, about 5, about 4, about 3, about 2, about 1 minute(s) following wounding or surgery, for example. Alternatively, the anti-connexin polynucleotide may be applied to an existing abnormal or excessive scar, e.g., a keloid or hypertrophic scar.

Conveniently, the composition is administered in a sufficient amount to downregulate expression of said connexin protein(s) for at least about 1-2 hours, at least about 2-4 hours, at least about 4-6 hours, at least about 6-8 hours, or about 24 hours post-administration.

According to an aspect, the anti-connexin polynucleotides may be administered topically or instilled or injected (at the site to be treated). In one aspect, the anti-connexin polynucleotides are combined with a pharmaceutically acceptable carrier or diluent to produce a pharmaceutical composition. Suitable carriers and diluents include isotonic saline solutions, for example phosphate-buffered saline. In another aspect, the composition may be formulated for intramuscular, subcutaneous, or transdermal administration.

Other suitable formulations include pluronic gel-based formulations, carboxymethylcellulose(CMC)-based formulations, and hydroxypropylmethylcellulose(HPMC)-based formulations. The composition may be formulated for any desired form of delivery, including topical, instillation, parenteral, intramuscular, subcutaneous, or transdermal administration. Other useful formulations include slow or delayed release preparations.

Where the anti-connexin agent is a nucleic acid, such as a polynucleotide, uptake of nucleic acids by mammalian cells is enhanced by several known transfection techniques, for example, those including the use of transfection agents. Such techniques may be used with certain anti-connexin agents, including polynucleotides. The formulation which is administered may contain such transfection agents. Examples of such agents include cationic agents (for example calcium phosphate and DEAE-dextran and lipofectants (for example lipofectam™ and transfectam™), and surfactants.

In one embodiment, the formulation further includes a surfactant to assist with polynucleotide cell penetration or the formulation may contain any suitable loading agent. Any suitable non-toxic surfactant may be included, such as DMSO. Alternatively a transdermal penetration agent such as urea may be included.

Optionally, the anti-connexin polynucleotide may be formulated with one or more therapeutic agents, agents useful for wound healing, and/or protein synthesis inhibitors. In certain embodiments, one, two three, four, five or six therapeutic agents may be used in combination. In certain embodiments, one, two three, four, five or six agents useful for wound healing may be used in combination. In certain embodiments, one, two, three, four, five or six protein synthesis inhibitors may be used in combination.

In one aspect, the one or more anti-connexin polynucleotides, either alone or in combination with one or more therapeutic agents and/or agents useful in wound healing are provided in the form of a wound dressing or matrix. In certain embodiments, the one or more anti-connexin polynucleotides (with or without one or more therapeutic agents, agents useful in wound healing and/or protein synthesis inhibitors) are provided in the form of a liquid, semi solid or solid composition for application directly, or the composition is applied to the surface of, or incorporated into, a solid contacting layer such as a dressing gauze or matrix. The wound dressing composition may be provided for example, in the form of a fluid or a gel. The one or more anti-connexin polynucleotides (with or without one or more therapeutic agents, agents useful in wound healing and/or protein synthesis inhibitors) may be provided in combination with conventional pharmaceutical excipients for topical application. Suitable carriers include: Pluronic gels, Polaxamer gels, Hydrogels containing cellulose derivatives, including hydroxyethyl cellulose, hydroxymethyl cellulose, carboxymethyl cellulose, hydroxypropylmethyl cellulose and mixtures thereof; and hydrogels containing polyacrylic acid (Carbopols). Suitable carriers also include creams/ointments used for topical pharmaceutical preparations, e.g., creams based on cetomacrogol emulsifying ointment. The above carriers may include alginate (as a thickener or stimulant), preservatives such as benzyl alcohol, buffers to control pH such as disodium hydrogen phosphate/sodium dihydrogen phosphate, agents to adjust osmolarity such as sodium chloride, and stabilizers such as EDTA.

The effective dose for a given subject preferably lies within the dose that is therapeutically effective for at least 50% of the population, and that exhibits little or no toxicity at this level.

The effective dose for a given subject or condition can be determined by routine experimentation or other methods known in the art or later developed. For example, in order to formulate a range of dosage values, cell culture assays and animal studies can be used. The dosage of such compounds preferably lies within the dose that is therapeutically effective for at least about 50% of the population, and that exhibits little or no toxicity at this level.

The effective dosage of each of the anti-connexin polynucleotides employed in the methods and compositions of the invention may vary depending on a number of factors including the particular anti-connexin polynucleotide employed, the mode of administration, the frequency of administration, the wound being treated, the severity of the wound being treated, the route of administration, the needs of a subject sub-population to be treated or the needs of the individual subject which different needs can be due to age, sex, body weight, relevant medical wound specific to the subject.

For example, in certain embodiments, the combined anti-connexin polynucleotide composition may be applied at about 0.05 micromolar (uM) to about 100 uM final concentration at the wound or adjacent to the wound site, preferably, the combined anti-connexin agent composition is applied at about 0.05 uM to about 50 uM final concentration, more preferably, the combined anti-connexin agent composition is applied at about 10 uM to about 30 uM final concentration, additionally, the combined anti-connexin agent composition is applied at about 8 uM to about 20 uM final concentration, alternatively, the combined anti-connexin agent composition is applied at about 10 uM to about 20 uM final concentration, even more preferably, the combined anti-connexin agent composition is applied at about 10 to about 15 uM final concentration. In certain other embodiment, the combined anti-connexin agent composition is applied at about 10 uM final concentration. In yet another embodiment, the combined anti-connexin agent composition is applied at about 15 uM final concentration.

Anti-connexin polynucleotide dose amounts include, for example, about 1, 2 3 4, or 5 micrograms, from about 5 to about 10 micrograms, from about 10 to about 15 micrograms, from about 15 to about 20 micrograms, from about 20 to about 30 micrograms, from about 30 to about 40 micrograms, from about 40 to about 50 micrograms, from about 50 to about 75 micrograms, from about 75 to about 100 micrograms, from about 100 micrograms to about 250 micrograms, and from 250 micrograms to about 500 micrograms. Dose amounts from about 0.5 to about 1.0 milligrams or more or also provided, as noted herein.

A suitable dose may be from about 0.001 to about 1 mg/kg body weight such as about 0.01 to about 0.4 mg/kg body weight. A suitable dose may however be from about 0.001 to about 0.1 mg/kg body weight such as about 0.01 to about 0.050 mg/kg body weight. Doses from about 1 to 100, 100-200, 200-300, 300-400, and 400-500 micrograms are appropriate, as well as doses from about 500-750 and from about 750-1000 micrograms. As noted herein, repeat applications are contemplated. Repeat applications are typically applied about once per week, or when wound-healing may appear to be stalled or slowing.

Alternatively, dosage of each anti-connexin polynucleotide may be based on the amount of anti-connexin polynucleotide per kg body weight of the patient. Suitable doses are from about 0.1 to about 1 mg/kg body weight such as about 1 to about 0.4 mg/kg body weight. A suitable dose may however be from about 0.001 to about 0.1 mg/kg body weight such as about 0.01 to about 0.050 mg/kg body weight. Other doses range from about 0.1 to about 1000 micrograms, and specifically included are all amounts in between as if written out herein. The doses may be administered in single or divided applications. The doses may be administered once, or application may be repeated. Still other dosage levels between about 1 nanogram (ng)/kg and about 1 mg/kg body weight per day of each of the agents described herein. In certain embodiments, the dosage of each of the subject compounds will generally be in the range of about 1 ng to about 1 microgram per kg body weight, about 1 ng to about 0.1 microgram per kg body weight, about 1 ng to about 10 ng per kg body weight, about 10 ng to about 0.1 microgram per kg body weight, about 0.1 microgram to about 1 microgram per kg body weight, about 20 ng to about 100 ng per kg body weight, about 0.001 mg to about 1 mg per kg body weight, about 0.01 mg to about 10 mg per kg body weight, or about 0.1 mg to about 1 mg per kg body weight. In certain embodiments, the dosage of each of the subject compounds will generally be in the range of about 0.001 mg to about 0.01 mg per kg body weight, about 0.01 mg to about 0.1 mg per kg body weight, about 0.1 mg to about 1 mg per kg body weight, or about 1 mg per kg body weight. If more than one anti-connexin polynucleotide is used, the dosage of each anti-connexin polynucleotide need not be in the same range as the other. For example, the dosage of one anti-connexin polynucleotide may be between about 0.01 mg to about 1 mg per kg body weight, and the dosage of another anti-connexin polynucleotide may be between about 0.1 mg to about 1 mg per kg body weight. As noted herein, repeat applications are contemplated. Repeat applications are typically applied about once per week, or when wound-healing may appear to be stalled or slowing.

Other useful doses range from about 1 to about 10 micrograms per square centimeter of scar (existing or predicted) or wound size. Certain doses will be about 1-2, about 1-5, about 2-4, about 5-7, and about 8-10 micrograms per square centimeter of scar (existing or predicted) or wound size. Other useful doses are greater than about 10 micrograms per square centimeter of scar (existing or predicted) or wound size, including about 15 micrograms per square centimeter of scar (existing or predicted) or wound size, about 20 micrograms per square centimeter of scar (existing or predicted) or wound size, about 25 micrograms per square centimeter of scar (existing or predicted) or wound size, about 30 micrograms per square centimeter of scar (existing or predicted) or wound size, about 35 micrograms per square centimeter of scar (existing or predicted) or wound size, about 40 micrograms per square centimeter of scar (existing or predicted) or wound size, about 50 micrograms per square centimeter of scar (existing or predicted) or wound size, and about 100 micrograms per square centimeter of scar (existing or predicted) or wound size. Other useful doses are about 150 micrograms per square centimeter of scar (existing or predicted) or wound size, about 200 micrograms per square centimeter of scar (existing or predicted) or wound size, about 250 micrograms per square centimeter of scar (existing or predicted) or wound size, or about 500 micrograms per square centimeter of scar (existing or predicted) or wound size. As noted herein, repeat applications are contemplated. Repeat applications are typically applied about once per week, or when wound-healing may appear to be stalled or slowing.

For example, in certain embodiments, the anti-connexin polynucleotide composition may be applied at about 0.01 micromolar (μM) or 0.05 μM to about 200 μM final concentration at the treatment site and/or adjacent to the treatment site. Preferably, the antisense polynucleotide composition is applied at about 0.05 μM to about 100 μM final concentration, more preferably, the anti-connexin polynucleotide composition is applied at about 0.05 μM to about 50 μM final concentration, and more preferably, the anti-connexin polynucleotide composition is applied at about 5-10 μM to about 30-50 μM final concentration. Additionally, the anti-connexin polynucleotide composition is applied at about 8 μM to about 20 μM final concentration, and alternatively the anti-connexin polynucleotide composition is applied at about 10 μM to about 20 μM final concentration, or at about 10 to about 15 μM final concentration. In certain other embodiments, the anti-connexin polynucleotide is applied at about 10 μM final concentration. In yet another embodiment, the anti-connexin polynucleotide composition is applied at about 1-15 μM final concentration. Anti-connexin polynucleotide dose amounts include, for example, about 0.1-1, 1-2, 2-3, 3-4, or 4-5 micrograms (μg), from about 5 to about 10 μg, from about 10 to about 15 μg, from about 15 to about 20 μg, from about 20 to about 30 μg, from about 30 to about 40 μg, from about 40 to about 50 μg, from about 50 to about 75 μg, from about 75 to about 100 μg, from about 100 μg to about 250 μg, and from 250 μg to about 500 μg. Dose amounts from 0.5 to about 1.0 milligrams or more or also provided, as noted above. Dose volumes will depend on the size of the site to be treated, and may range, for example, from about 25-100 μL to about 100-200 μL, from about 200-500 μL to about 500-1000 μL (microliter) doses are also appropriate for larger treatment sites. As noted herein, repeat applications are contemplated. Repeat applications are typically applied about once per week, or when wound-healing may appear to be stalled or slowing.

Conveniently, the anti-connexin polynucleotide is administered in a sufficient amount to downregulate expression of a connexin protein, or modulate gap junction formation for at least about 0.5 to 1 hour, at least about 1-2 hours, at least about 2-4 hours, at least about 4-6 hours, at least about 6-8 hours, at least about 8-10 hours, at least about 12 hours, or at least about 24 hours post-administration.

The dosage of each of the anti-connexin polynucleotides in the compositions and methods of the subject invention may also be determined by reference to the concentration of the composition relative to the size, length, depth, area or volume of the area to which it will be applied. For example, in certain topical and other applications, e.g., instillation, dosing of the pharmaceutical compositions may be calculated based on mass (e.g. micrograms) of or the concentration in a pharmaceutical composition (e.g. μg/μl) per length, depth, area, or volume of the area of application.

The initial and any subsequent dosages administered will depend upon factors noted herein. Depending on the oligonucelotide, the dosage and protocol for administration will vary, and the dosage will also depend on the method of administration selected, for example, local or topical administration.

Agents useful for wound healing suitable for the preparation of the pharmaceutical compositions described herein may be prepared and administered using methods as known in the art (see, for example, U.S. Pat. Nos. 7,098,190, 6,319,907, 6,331,298, 6,387,364, 6,455,569, 6,566,339, 6,696,433, 6,855,505, 6,900,181, 7,052,684 and EP1100529 B1. The concentration of each anti-connexin polynucleotide and agents useful for wound healing need not be in the same range as the other. Other amounts will be known to those of skill in the art and readily determined. For example, suitable combination dosages and formulations in accordance with various aspects and embodiments as described herein may be administered according to the dosing regimen as described in U.S. Pat. No. 6,903,078 to Lewis entitled “Combination PDGF, KGF, IGF, and IGFBP for wound healing.”

The initial and any subsequent dosages administered will depend upon the subject's age, weight, condition, and the scar being treated. Depending on the agent useful for wound healing, the dosage and protocol for administration will vary, and the dosage will also depend on the method of administration selected, for example, local or systemic administration.

The agent useful for wound healing may be applied internally or externally, and may be directed towards any tissue exhibiting a wound. For topical administration of IGF, for example, a zinc oxide formulation can be applied, which induces the local production of IGF, as described in Tarnow et al, Scand J. Plast Reconstr Hand Surg. 28: 255-259 (1994). An effective dose of PDGF has been reported to be 5 ng/mm² or higher when applied topically as described in U.S. Pat. No. 4,861,757, and at least 1 ng/ml local concentration of an isoform of PDGF (for example, PDGF-AA, PDGF-BB, or PDGF-AB), up to about 30 ng/ml local concentration applied to a population of fibroblasts as described in Lepisto et al., Biochem Biophys Res. Comm 209: 393-399 (1995). PDGF can be administered in a carboxymethylcellulose gel formulation at concentrations of about 10 μg/gm to about 500 μg/gm of gel, about 20 μg/gm to about 200 μg/gm, and about 30 μg/gm to about 100 μg/gm of gel, optimally about 100 μg/gm of gel. Efficacy of PDGF has been achieved within the range of about 3 μg/ml solution to about 300 μg/ml of solution administered.

About 50 μl of KGF of a concentration of about 5 μg/ml may be effective for wound healing by topical application to epithelial tissue as described in Sotozono et al, Invest. Opthal. Vis. Science 36: 1524-29 (1995). As described in U.S. Pat. No. 4,861,757, an effective amount of IGF when co-administered with PDGF is in the range of at least 2.5 ng/mm² to about 5 ng/mm², with a ratio of PDGF to IGF in the range of about 1:10 to about 25:1 weight to weight, with the most effective ratios being PDGF to IGF of about 1:1 to about 2:1 weight to weight. IGFBP administered in combination with IGF has been shown to increase wound healing at dose levels of about 5 μg of IGF with about 1.5 μg of phosphorylated IGFBP in a molar ration of about 11:1 IGF:IGFBP, as described in Jyung et al, Surgery 115:233-239 (1994).

For administration of polypeptide therapeutics, for example, PDGF, KGF, IGF and IGFBP polypeptides, the dosage can be in the range of about 5 μg to about 50 μg/kg of tissue to which the application is directed, also about 50 μg to about 5 mg/kg, also about 100 μg to about 500 μg/kg of tissue, and about 200 to about 250 μg/kg. For polynucleotide therapeutics, for example in a gene therapy administration protocol, depending on the expression strength the polynucleotide in the subject, for tissue targeted administration, vectors containing expressible constructs including PDGF, KGF, IGF, and IGFBP coding sequences can be administered in a range of about 100 ng to about 200 mg of DNA for local administration in a gene therapy protocol, also about 500 ng to about 50 mg, also about 1 μg to about 2 mg of DNA, about 5 μg of DNA to about 500 μg of DNA, and about 20 μg to about 100 μg during a local administration in a gene therapy protocol, and about 250 μg, per injection or administration. Factors such as method of action and efficacy of transformation and expression are therefore considerations that will effect the dosage required for ultimate efficacy for administration of DNA therapeutics. Where greater expression is desired, over a larger area of tissue, larger amounts of DNA or the same amounts re-administered in a successive protocol of administrations, or several administrations to different adjacent or close tissue portions of for example, a wound site may be required to effect a positive therapeutic outcome.

Therapeutic agents and protein synthesis inhibitors suitable for the preparation of the pharmaceutical compositions described herein may be formulated and administered using methods as known in the art. The initial and any subsequent dosages administered will depend upon the subject's age, weight, condition, and the disease, wound, disorder or biological condition being treated. Depending on the therapeutic, the dosage and protocol for administration will vary, and the dosage will also depend on the method of administration selected, for example, local or systemic administration.

As noted herein, the doses of either an anti-connexin polynucleotides or another agent administered in combination can be adjusted down from the doses administered when given alone.

The combined use of several anti-connexin polynucleotides may reduce the required dosage for any individual component because the onset and duration of effect of the different components may be complementary. In a preferred embodiment, the combined use of one or more anti-connexin polynucleotides and one or more therapeutic agents, agents useful for wound healing, and/or protein synthesis inhibitors have an additive, synergistic or super-additive effect.

In some cases, the combination of one or more anti-connexin polynucleotides and one or more therapeutic agents, one or more agents useful for wound healing, and/or one or more protein synthesis inhibitors have an additive effect. In other cases, the combination can have greater-than-additive effect. Such an effect is referred to herein as a “supra-additive” effect, and may be due to synergistic or potentiated interaction.

The term “supra-additive promotion of wound healing” refers to a mean wound healing produced by administration of a combination of an anti-connexin polynucleotide and one or more therapeutic agents, agents useful for wound healing, and/or protein synthesis inhibitors, is statistically significantly higher than the sum of the wound healing produced by the individual administration of either any of the agents alone. Whether produced by combination administration of an anti-connexin polynucleotide and one or more therapeutic agents, agents useful for wound healing, and/or protein synthesis inhibitors is “statistically significantly higher” than the expected additive value of the individual compounds may be determined by a variety of statistical methods as described herein and/or known by one of ordinary skill in the art. The term “synergistic” refers to a type of supra-additive inhibition in which both the anti-connexin polynucleotide and one or more therapeutic agents, agents useful for wound healing and/or protein synthesis inhibitors individually have the ability to promote wound healing or reduce scarring. The term “potentiated” refers to type of supra-additive effect in which one of the anti-connexin polynucleotide or one or more therapeutic agents, agents useful for wound healing, and/or protein synthesis inhibitors individually has the increased ability to reduce excess scarring.

In general, potentiation may be assessed by determining whether the combination treatment produces a mean reduction in excess scar formation in a treatment group that is statistically significantly supra-additive when compared to the sum of the mean scar formation produced by the individual treatments in their treatment groups respectively. The mean in reduced scar formation may be calculated as the difference between control group and treatment group mean wound healing. The fractional decrease in scar formation, “fraction affected” (Fa), may be calculated by dividing the treatment group scar formation by control group mean scar formation. Testing for statistically significant potentiation requires the calculation of Fa for each treatment group. The expected additive Fa for a combination treatment may be taken to be the sum of mean Fas from groups receiving either element of the combination. The Two-Tailed One-Sample T-Test, for example, may be used to evaluate how likely it is that the result obtained by the experiment is due to chance alone, as measured by the p-value. A p-value of less than 0.05 is considered statistically significant, that is, not likely to be due to chance alone. Thus, Fa for the combination treatment group must be statistically significantly higher than the expected additive Fa for the single element treatment groups to deem the combination as resulting in a potentiated supra-additive effect.

Whether a synergistic effect results from a combination treatment may be evaluated by the median-effect/combination-index isobologram method (Chou, T., and Talalay, P. (1984) Ad. Enzyme Reg. 22:27-55). In this method, combination index (CI) values are calculated for different dose-effect levels based on parameters derived from median-effect plots of the anti-connexin polynucleotide alone, the one or more therapeutics agents, agents useful for wound healing and/or protein synthesis inhibitors alone, and the combination of the two at fixed molar ratios. CI values of & lt; 1 indicate synergy, CI-1 indicates an additive effect, and CP1 indicates an antagonistic effect. This analysis may be performed using computer software tools, such as CalcuSyn, Windows Software for Dose Effect Analysis (Biosoft(D, Cambridge UK).

Any method known or later developed in the art for analyzing whether a supra-additive effect exists for a combination therapy is contemplated for use in screening for suitable anti-connexin polynucleotides for use in combination with one or more therapeutic agents, agents useful for wound healing, and/or protein synthesis inhibitors.

In another preferred embodiment, the combined use of one or more anti-connexin polynucleotides and one or more therapeutic agents, agents useful for wound healing, and/or protein synthesis inhibitors reduces the effective dose of any such agent compared to the effective dose when said agent administered alone. In certain embodiments, the effective dose of the agent when used in combination with one or more anti-connexin polynucleotides is about 1/15 to about ½, about 1/10 to about ⅓, about ⅛ to about ⅙, about ⅕, about ¼, about ⅓ or about ½ the dose of the agent when used alone.

In another preferred embodiment, the combined use of one or more anti-connexin polynucleotides and one or more therapeutic agents, agents useful for wound healing, and/or protein synthesis inhibitors reduces the frequency in which said agent is administered compared to the frequency when said agent is administered alone. Thus, these combinations allow the use of lower and/or fewer doses of each agent than previously required to achieve desired therapeutic goals.

The doses may be administered in single or divided applications. The doses may be administered once, or application may be repeated.

One or more anti-connexin polynucleotides, either alone or in combination with one or more therapeutic agents and/or one or more agents useful in wound healing, may be administered by the same or different routes. The various agents of the invention can be administered separately at different times during the course of therapy, or concurrently in divided or single combination forms.

Preferably one or more anti-connexin polynucleotides useful in the treatment of abnormal or excess scarring are delivered by topical administration (peripherally or directly to a site), including but not limited to topical administration using solid supports (such as dressings and other matrices) and medicinal formulations (such as gels, mixtures, suspensions and ointments). In one embodiment, the solid support comprises a biocompatible membrane or insertion into a treatment site. In another embodiment, the solid support comprises a dressing or matrix. In one embodiment of the invention, the solid support composition may be a slow release solid support composition, in which the one or more anti-connexin polynucleotides useful for wound healing is dispersed in a slow release solid matrix such as a matrix of alginate, collagen, or a synthetic bioabsorbable polymer. Preferably, the solid support composition is sterile or low bio-burden. In one embodiment, a wash solution comprising one or more anti-connexin polynucleotides can be used.

The delivery of one or more anti-connexin polynucleotides (with or without one or more therapeutic agents, agents useful for wound healing, and/or protein synthesis inhibitors) may occur over a period of time, in some instances for about 0.5 hours, 1-2 hours, about 2-4 hours, about 4-6 hours, about 6-8, or about 24 hours or longer, may be a particular advantage in more severe wounds. In some instances, cell loss may extend well beyond the site of a procedure to surrounding cells. Such loss may occur within 24 hours of the original procedure and is mediated by gap junction cell-cell communication. Administration of anti-connexin polynucleotide(s) will modulate communication between the cells and minimize additional cell loss or injury or consequences of injury.

While the delivery period will be dependent upon both the site at which the downregulation is to be induced and the therapeutic effect which is desired, continuous or slow-release delivery for about 0.5 hours, about 1-2 hours, about 2-4 hours, about 4-6 hours, about 6-8, or about 24 hours or longer is provided. In accordance with the present invention, this maybe achieved by inclusion of the anti-connexin polynucleotides (with or without one or more therapeutic agents, agents useful for wound healing and/or protein synthesis inhibitors) in a formulation together with a pharmaceutically acceptable carrier or vehicle, particularly in the form of a formulation for continuous or slow-release administration.

The routes of administration and dosages described herein are intended only as a guide since a skilled physician will determine the optimum route of administration and dosage for any particular subject and wound.

In one embodiment of the invention, the dressing composition may be a slow release solid composition, in which the one or more anti-connexin polynucleotides and/or one or more anti-scarring factors or agents is dispersed in a slow release solid matrix such as a matrix of alginate, collagen, or a synthetic bioabsorbable polymer. Preferably, the dressing composition is sterile or low bio-burden.

Optionally, one or more other, anti-scarring factors or agents (e.g., peptides, proteolytic inhibitors, extracellular matrix components, fragments and peptides, steroids, cytokines, oxygen donators or vitamins) may also be used in the manufacture of the medicament, pharmaceutical compositions and combined preparations according to the invention. Such anti-scarring agents may also be used in the method of the present invention. The inclusion of these agents may allow enhanced prevention or treatment of abnormal or excessive scars. Such additional anti-scarring factors or agents may be administered separately, simultaneously or sequentially, or in combination with the one or more anti-connexin polynucleotides.

Thus, optionally, an anti-connexin polynucleotide or compounds may be formulated with one or more therapeutic agents, anti-scarring or wound healing agents, and/or gap junction modifying agents. Therapeutic agents include, for example, anti-infectives, anaesthetics, analgesics, antibiotics, narcotics, and steroidal and non-steroidal anti-inflammatory agents. In certain embodiments, one, two three, four, five or six therapeutic agents may be used in combination.

Any of the methods of treating a subject having or suspected of having or a disease, disorder, and/or wound, referenced or described herein may utilize the administration of any of the doses, dosage forms, formulations, and/or compositions herein described.

### Dressings and Matrices

In one aspect, the one or more anti-connexin polynucleotides, either alone or in combination with one or more therapeutic agents, agents useful for wound healing, and/or protein synthesis inhibitors are provided in the form of a dressing or matrix. In certain embodiments, the one or more agents of the invention are provided in the form of a liquid, semi solid or solid composition for application directly, or the composition is applied to the surface of, or incorporated into, a solid contacting layer such as a dressing gauze or matrix. The dressing composition may be provided for example, in the form of a fluid or a gel. The one or more anti-connexin polynucleotides, either alone or in combination with one or more therapeutic agents, agents useful for wound healing, and/or protein synthesis inhibitors may be provided in combination with conventional pharmaceutical excipients for topical application. Suitable carriers include: Pluronic gels, Polaxamer gels, Hydrogels containing cellulose derivatives, including hydroxyethyl cellulose, hydroxymethyl cellulose, carboxymethyl cellulose, hydroxypropylmethyl cellulose and mixtures thereof; and hydrogels containing polyacrylic acid (Carbopols). Suitable carriers also include creams/ointments used for topical pharmaceutical preparations, e.g., creams based on cetomacrogol emulsifying ointment. The above carriers may include alginate (as a thickener or stimulant), preservatives such as benzyl alcohol, buffers to control pH such as disodium hydrogen phosphate/sodium dihydrogen phosphate, agents to adjust osmolarity such as sodium chloride, and stabilizers such as EDTA.

Dressings and Matrices

Suitable dressings or matrices for the treatment of abnormal or excessive scars as described herein may include, for example, the following in combination with one or more anti-connexin polynucleotides, alone or in conjunction with other anti-scarring or wound-healing agents, for example:

In one embodiment one or more anti-connexin polynucleotides, for example a connexin 43 antisense polynucleotide, preferably a connexin 43 antisense oligodeoxynucleotide, is administered on a natural or synthetic matrix.

Suitable dressings or matrices may include, for example, the following with one or more anti-connexin polynucleotides (with or without one or more therapeutic agents, agents useful for wound healing, and/or protein synthesis inhibitors). An anti-connexin 43 oligonucleotide is preferred, for example an anti-connexin 43 antisense oligonucleotide:

1) Absorptives: suitable absorptives may include, for example, absorptive dressings, multi-layer anti-connexin anti-scarring covers which can provide, for example, a semi-adherent quality or a non-adherent layer, combined with highly absorptive layers of fibers, such as for example, cellulose, cotton or rayon. Alternatively, absorptives may be used as a primary or secondary dressing to manage abnormal or excessive scarring.

2) Alginates: suitable alginates include, for example, dressings that are non-woven, non-adhesive pads and ribbons composed of natural polysaccharide fibers or xerogel derived from seaweed. Suitable alginates dressings may, for example, form a moist gel through a process of ion exchange upon contact with exudate. In certain embodiments, anti-connexin anti-scarring alginate dressings are designed to be soft and conformable, easy to pack, tuck or apply over irregular-shaped areas. In certain embodiments, alginate dressings may be used with a second dressing.

3) Antimicrobial Dressings: suitable antimicrobial dressings may include, for example, anti-connexin anti-scarring dressings that can facilitate delivery of bioactive agents, such as, for example, silver and polyhexamethylene biguanide (PHMB), to maintain efficacy against infection, where this is needed or desirable. In certain embodiments, suitable antimicrobial dressings may be available as for example, as sponges, impregnated woven gauzes, film dressings, absorptive products, island dressings, nylon fabric, non-adherent barriers, or a combination of materials.

4) Biological & Biosynthetics: suitable biological dressings or biosynthetic dressings may include, for example, gels, solutions or semi-permeable sheets derived from a natural source. In certain embodiments, a gel or solution is applied to the area in which abnormal or excessive scar formation is to be prevented and covered with an anti-connexin anti-scarring dressing for barrier protection. In another embodiment, a sheet is placed in situ which may act as membrane, remaining in place after a single application to prevent or treat abnormal or excessive scars.

5) Collagens: suitable collagen dressings may include, for example, gels, pads, particles, pastes, powders, sheets or solutions derived from for example, bovine, porcine or avian sources or other natural sources or donors. In certain embodiments, the collagen dressing may interact with wound site exudate to form a gel. In certain embodiments, collagen dressing may be used in combination with a secondary dressing.

6) Composites: suitable composite anti-connexin anti-scarring dressings may include, for example, covers dressings that combine physically distinct components into a single product to provide multiple functions, such as, for example, a bacterial barrier, absorption and adhesion. In certain embodiment, the anti-connexin anti-scarring composite dressings are comprised of, for example, multiple layers and incorporate a semi-or non-adherent pad. In certain other embodiments, the composite dressing may function as for example, either a primary or a secondary dressing on a wide variety of areas in which abnormal or excessive scars are to be prevented or treated and in yet other embodiments, the dressing may be used in combination with another topical pharmaceutical composition.

7) Contact Layers: suitable anti-connexin anti-scarring contact layer dressings may include, for example, thin, non-adherent sheets placed on an area to protect tissue from for example, direct contact with other agents or dressings applied to the area in which abnormal or excessive scars are to be prevented or treated. In certain embodiments, contact layers may be deployed to conform to the shape of the area in which abnormal or excessive scars are be prevented and treated and are porous to allow exudate to pass through for absorption by an overlying, secondary dressing. In yet another embodiment, the anti-connexin anti-scarring contact layer dressing may include, for example, non-immunogenic and/or anti-adhesive gauzes, films, sheets, dressings, sponges, or wraps to be placed in situ.

8) Elastic Bandages: suitable elastic bandages may include, for example, dressings that stretch and conform to the body contours. In certain embodiment, the fabric composition may include for example, cotton, polyester, rayon or nylon. In certain other embodiments, the elastic bandage may for example, provide absorption as a second layer or dressing, to hold a cover in place, to apply pressure or to cushion an area in which abnormal or excessive scars are to be prevented or treated.

9) Foams: suitable anti-connexin anti-scarring foam dressings may include, for example, sheets and other shapes of foamed polymer solutions (including polyurethane) with small, open cells capable of holding fluids. Exemplary foams may be for example, impregnated or layered in combination with other materials. In certain embodiments, the absorption capability may be adjusted based on the thickness and composition of the foam at the site where abnormal or excessive scars are to be prevented or treated. In certain other embodiments, the area in contact with the area where abnormal or excessive scars are to be prevented or treated may be non-adhesive for easy removal. In yet another embodiment, the foam may be used in combination with an adhesive border and/or a transparent film coating that can serve as an anti-infective barrier.

10) Gauzes & Non-Woven dressings: suitable anti-connexin anti-scarring gauze dressings and woven dressings may include, for example, dry woven or non-woven sponges and wraps with varying degrees of absorbency. Exemplary fabric composition may include, for example, cotton, polyester or rayon. In certain embodiments, gauzes and non-woven dressing may be available sterile or non-sterile in bulk and with or without an adhesive border. Exemplary anti-connexin anti-scarring gauze dressings and woven dressings may be used for cleansing, packing and covering a variety of wound areas where abnormal or excessive scars are to be prevented or treated.

11) Hydrocolloids: suitable anti-connexin anti-scarring hydrocolloid dressings may include, for example, wafers, powders or pastes composed of gelatin, pectin or carboxymethylcellulose. In certain embodiments, wafers are self-adhering and available with or without an adhesive border and in a wide variety of shapes and sizes. Exemplary hydrocolloids are useful on areas that require contouring. In certain embodiments, powders and pastes hydrocolloids may use used in combination with a secondary dressing.

12) Hydrogels (Amorphous): suitable anti-connexin anti-scarring amorphous hydrogel dressings may include, for example, formulations of water, polymers and other ingredients with no shape, designed to donate moisture and to maintain a moist healing environments and or to rehydrate the area where abnormal or excessive scars are to be prevented or treated. In certain embodiments, hydrogels may be used in combination with a secondary dressing cover.

13) Hydrogels: Impregnated Dressings: suitable impregnated anti-connexin anti-scarring hydrogel dressings may include, for example, gauzes and non-woven sponges, ropes and strips saturated with an amorphous hydrogel. Amorphous hydrogels may include for example, formulations of water, polymers and other ingredients with no shape, designed to donate moisture to a dry wound and to maintain a moist healing environment.

14) Hydrogel Sheets: suitable anti-connexin anti-scarring hydrogel sheets may include for example, three-dimensional networks of cross-linked hydrophilic polymers that are insoluble in water and interact with aqueous solutions by swelling. Exemplary hydrogels are highly conformable and permeable and can absorb varying amounts of drainage, depending on their composition. In certain embodiment, the hydrogel is non-adhesive against the area in which abnormal or excessive scars are to be prevented or treated for easy removal.

15) Impregnated Dressings: suitable anti-connexin anti-scarring impregnated dressings may include, for example, gauzes and non-woven sponges, ropes and strips saturated with a solution, an emulsion, oil, gel or some other pharmaceutically active compound or carrier agent, including for example, saline, oil, zinc salts, petrolatum, xeroform and scarlet red as well as the anti-keloid/anti-hypertrophic scar compounds described herein.

16) Silicone Gel Sheets: suitable anti-connexin anti-scarring silicone gel sheet dressings may include, for example, soft wound covers composed of cross-linked polymers reinforced with or bonded to mesh or fabric.

17) Solutions: suitable anti-connexin anti-scarring liquid dressings may include, for example, mixtures of multiprotein material and other elements found in the extracellular matrix. In certain embodiments, exemplary solutions may be applied to a wound surface after scar removal and cleansing and then covered with an absorbent dressing or a nonadherent pad.

18) Transparent Films: suitable anti-connexin anti-scarring transparent film dressings may include polymer membranes of varying thickness coated on one side with an adhesive. In certain embodiment, transparent films are impermeable to liquid, water and bacteria but permeable to moisture vapor and atmospheric gases. In certain embodiment, the transparency allows visualization of the wound.

19) Wound Fillers: suitable anti-connexin anti-scarring wound filler dressings may include, for example, beads, creams, foams, gels, ointments, pads, pastes, pillows, powders, strands or other formulations. In certain embodiments, fillers are non-adherent and may include a time-released antimicrobial. Exemplary fillers may be useful to maintain a moist environment, manage exudate, and for treatment of for example, partial- and full-thickness wounds, infected wounds, draining wounds and deep wounds that require packing.

Thus, in accordance with the invention, there are provided formulations by which cell-cell communication can be regulated or downregulated in a transient and site-specific manner. The formulations therefore have application in methods of therapy and in other treatments.

In instances of tissue damage which may produce excessive scarring and/or abnormal or excessive scars, the formulations of the invention will be effective in both preventing abnormal or excessive scars, such as keloids and/or, hypertrophic scars, atrophic scars, and widespread scars decreasing severity and promoting the healing process where needed. The formulations therefore will have benefit in the prevention and/or treatment of excessive and abnormal or excessive scarring and of keloids and/or hypertrophic scars, whether the result of external trauma, surgical intervention or disease state, for example.

Methods of Treatment

Disorders to be Treated

The anti-connexin polynucleotides can be used to prevent or inhibit excessive and/or abnormal or excessive scar formation, especially hypertrophic scars and keloid scars, widespread scars and atrophic scars. Other conditions which should be beneficially treated using the anti-connexin polynucleotides include prevention of excessive and/or abnormal or excessive scarring following transplantation, cirrhosis of the liver, pulmonary fibrosis following acute respiratory distress syndrome or other pulmonary fibrosis of the newborn, and implantation of temporary prosthetics.

The method of the present invention can be used to minimize or prevent scar formation, such as hypertrophic wounds, keloids and excessive burn scarring, atrophic scars, and widespread scars, in humans or other mammals, particularly those individuals prone to excessive scarring. An anti-connexin polynucleotide, alone or in combination with or followed by another anti-scarring or wound-healing agent, can be applied to a presently existing abnormal or excessive scar, with or without scar revision surgery, to reverse the scarring process and essentially eliminate or reduce the scar tissue. The present invention can be used therapeutically to control diseases, conditions and procedures associated with excessive scarring.

Treatment Regimes

In one embodiment, the anti-connexin polynucleotide or other composition(s) of the invention is typically be administered either at the time of an injury or a surgery or shortly thereafter. Alternatively, it may be applied to an existing abnormal or excessive scar or to a wound which shows excessive scarring during healing.

The anti-connexin polynucleotide is administered in a dosage and in a regimen that does not prevent wound healing, but does decrease the amount of blood vessel growth at the wound site to prevent or decrease. It may also be administered in a dosage and in a regimen that prevents or decreases formation of high density cellular and connective tissue within the scar or outside of the wound area (keloids). In order to have increased levels of cells and deposited connective tissue one must have an increased nutritional supply via vascularization. Dosages will typically be in the same range as used for inhibition of tumor growth, but administered to a different class of subjects and for different time periods, since wound healing typically occurs over a much shorter time. Moreover, when administered topically or in a sustained release formulation, the dosage may be lower in order not to prevent wound healing.

This invention pertains to a method for minimizing or preventing excessive scar formation, particularly hypertrophic wound healing disorders, such as hypertrophic scars and keloids. Specifically, the method comprises administering an effective amount of an anti-connexin polynucleotide to a wound site for a period of time sufficient to minimize the scar, or to prevent the formation of a hypertrophic scar.

This invention pertains to a method for minimizing or preventing excessive and/or abnormal or excessive scar formation, particularly hypertrophic wound healing disorders, such as hypertrophic scars and keloids, as well as atrophic scars, and widespread scars. Specifically, the method comprises administering an effective amount of an anti-connexin polynucleotide to a wound site for a period of time sufficient to minimize the scar, or to prevent the formation of an abnormal or excessive scar.

In one embodiment, the anti-connexin polynucleotide can be administered alone or in combination with a protein synthesis inhibitor (such as a steroid) as part of a complete therapeutic regimen. For example, the steroid can be selected from the corticosteroids and glucocorticosteroids, such as triamcinolone acetonide. For similar purposes vitamin E can be co-administered. The anti-connexin polynucleotide is applied to the wound site, such as by injecting it directly onto or into a scar or topically applying it or instilling it onto or into the wound site. If a steroid or vitamin E is used in conjunction with the anti-connexin polynucleotide, the steroid can be co-administered or applied subsequently to the wound site, preferably within one to two days or within a two week time period. The steroid is also administered directly to the wound site; it may be injected or topically applied. In whatever manner they are administered, the anti-connexin polynucleotide and/or the steroid can be admixed with a pharmaceutically acceptable vehicle to facilitate localization of the agent to the wound site. Similarly, a therapeutic agent (e.g. non-steroidal anti-inflammatory agent) can be co-administered with the anti-connexin polynucleotide. The compounds may be put into sustained release formulation capsules to provide continuous treatment at therapeutic doses and without systemic side effects.

In one embodiment, abnormal or excessive scar content can be minimized by administering an effective amount of an anti-connexin polynucleotide to a hypertrophic or other abnormal or excessive wound site. The anti-connexin polynucleotide may be administered alone, or in combination with or followed by the administering of a protein synthesis inhibitor (e.g., steroid). For example, a steroid can be co-administered with the anti-connexin polynucleotide or applied separately, preferably within a two-week interval following the application of the anti-connexin polynucleotide. Treatment of the wound site with the anti-connexin polynucleotide, with or without the steroid, should continue for a period of time sufficient to minimize the abnormal or excessive scarring area. Suitable anti-connexin polynucleotides include those described herein and include, but are not limited to compounds, such as antisense oligonucleotides. The amount of anti-connexin polynucleotide which can be effectively administered is dependent upon the type of anti-connexin polynucleotide used and the scar or the scar site to be treated, and can be ascertained by monitoring the scar or scar site during treatment. The amount can be adjusted accordingly depending upon the scar or scar site. Effective amounts of anti-connexin polynucleotides can be in approximately 10 μM and 1 mM range. Steroids which may be used include, but are not limited to; corticosteroids and glucocorticosteroids, such as triamcinolone acetonide (also known as KENALOG™), and Vitamin E (α-tocopherol) (Ehrlich et al. 1972, Ann. Surg. 75:235). The amount of steroid which can be effectively administered will depend upon the type of steroid used. The effects of anti-connexin polynucleotide treatment, with and without steroids, on various types of wound scars are illustrated in the Examples.

In one embodiment any one of the methods of treatment described herein further comprises administration one or more therapeutic agents, agents useful for wound healing, and/or protein synthesis inhibitors. When not administered as a fixed combination, preferred methods include the sequential administration of one or more anti-connexin polynucleotides and one or more therapeutic agents, agents useful for wound healing and/or protein synthesis inhibitors. Preferably, the polynucleotides and agents are administered sequentially within at least about one-half hour of each other. The polynucleotides and agents may also be administered with about one hour of each other, with about one day to about one week of each other, or as otherwise deemed appropriate. Preferably, the anti-connexin polynucleotide is administered first.

In another embodiment for treatment and/or prevention of abnormal or excessive or excess scarring, either or both of the one or more anti-connexin polynucleotides and one or more therapeutic agents, agents useful for wound healing, and/or protein synthesis inhibitors are provided in amounts or doses that are less that those used when the polynucleotides or agents are administered alone, i.e., when they are not administered in combination, either physically or in the course of treatment of a wound. Such lesser amounts of agents administered are typically from about one-twentieth to about one-tenth the amount or amounts of the agent when administered alone, and may be about one-eighth the amount, about one-sixth the amount, about one-fifth the amount, about one-fourth the amount, about one-third the amount, and about one-half the amount when administered alone.

The method of administering an acceptable dose of anti-connexin polynucleotide to treat or prevent abnormal or excessive scarring is dependent upon the location of the wound and the extent of scarring. In particular, the anti-connexin polynucleotide, either alone or in combination with a pharmaceutically acceptable vehicle, can be topically applied to the surface of the scar or scar site; it can be injected into the scar or scar site; or it can be incorporated into a controlled release polymer or other matrix and surgically implanted in a scar or scar site to be treated. Surgical implantation is advantageous for treating or preventing larger abnormal or excessive scars or scar sites. This permits the anti-connexin polynucleotide to be localized in the scar or scar site without adversely affecting the patient or releasing excessive amounts of the drug into the circulation. If a steroid is used in conjunction with, or following, the anti-connexin polynucleotide, the acceptable dose of steroid may be administered through various methods. For example, the steroid, either alone or in combination with a pharmaceutically acceptable vehicle, can be topically applied to the surface of the wound site; injected into the wound site; or incorporated into a controlled release matrix and surgically implanted into the region to be treated.

Depolymerization of cycloskeletal proteins leading to alteration of cell shape and matrix degradation can be regulated using the methods of this invention. Secondary to this, the invention can be used to regulate and block exocytosis. In particular, fibroblasts are contacted with an effective amount of a calcium antagonist sufficient to degrade the matrix and retard exocytosis to a desired degree. The method of contacting the anti-connexin polynucleotides to the fibroblast cells of interest and the effective amount of these drugs are described above.

### Compositions

The present invention is directed to pharmaceutical compositions and formulations useful in treating or preventing abnormal or excess scarring (e.g. keloid or hypertrophic scarring or other abnormal or excessive scarring), wherein the composition or formulation comprises therapeutically effective amounts of one or more anti-connexin polynucleotides, such as a connexin antisense polynucleotide.

In one preferred form, the composition useful in treating or preventing abnormal or excess scarring (e.g. keloid or hypertrophic scarring or other abnormal or excessive scarring), contains one or more anti-connexin polynucleotides, for example a connexin antisense polynucleotide, to the mRNA of one connexin protein only. Most preferably, this connexin protein is connexin 43.

Alternatively, the compositions useful in treating or preventing abnormal or excess scarring (e.g. keloid or hypertrophic scarring or other abnormal or excessive scarring), may comprise polynucleotides to more than one connexin protein. Preferably, one of the connexin proteins to which polynucleotides are directed is connexin 43. Other connexin proteins to which oligodeoxynucleotides are directed may include, for example, connexins 26, 30, 31.1, 32, and 37. Suitable exemplary polynucleotides (and ODNs) directed to various connexins are set forth in Table 1.

Many aspects of the invention are described with reference to oligodeoxynucleotides. However it is understood that other suitable polynucleotides (such as RNA polynucleotides) may be used in these aspects. Other anti-connexin oligonucleotides are RNAi and siRNA oligonucleotides.

Accordingly, in one aspect, the invention provides compositions for use in therapeutic treatment for treating or preventing abnormal or excess scarring (e.g. keloid or hypertrophic scarring or other abnormal or excessive scarring), which comprises at least one anti-connexin polynucleotide, preferably an anti-connexin 43 polynucleotide. In a preferred embodiment, the composition further comprises a pharmaceutically acceptable carrier or vehicle. In another embodiment, the composition further comprises one or more therapeutic agents, one or more agents useful for wound healing and/or one or more protein synthesis inhibitors.

### Kits, Medicaments and Articles of Manufacturer

In one aspect, the invention provides a kit for treating or preventing abnormal or excess scarring (e.g. keloid or hypertrophic scarring or other abnormal or excessive scarring). The kit may include one or more compositions described herein. For example, the kit may include a composition comprising an effective amount of one or more anti-connexin polynucleotides, e.g., an anti-connexin 43 polynucleotides, effective for the treatment of a subject having, at risk for, or predisposition to abnormal or excess scarring. In one embodiment, the kit comprises a composition that comprises an effective amount of one or more polynucleotide homologues effective for the treatment of a subject having, at risk for, or predisposition to a fibrotic disease, disorder or condition. In another embodiment, the kit further comprises a second vessel comprising one or more of the following: therapeutic agent, agent useful for wound healing, and/or protein synthesis inhibitor (e.g. steroid or Vitamin E).

Optionally, one or more anti-connexin polynucleotides may also be used in the manufacture of the medicament useful in treating or preventing abnormal or excess scarring (e.g. keloid or hypertrophic scarring or other abnormal or excessive scarring). In one embodiment, the medicament comprises a therapeutically effective amount of an anti-connexin polynucleotide, preferably an anti-connexin 43 polynucleotide, and a pharmaceutically acceptable carrier.

In another aspect, the invention includes an article of manufacture useful in treating or preventing abnormal or excess scarring (e.g. keloid or hypertrophic scarring or other abnormal or excessive scarring), comprising a vessel containing an effective amount of one or more anti-connexin polynucleotides, e.g., an anti-connexin 43 polynucleotide, and instructions for use, including use for the treatment of a subject having, at risk for, or predisposition to abnormal or excess scarring. In one embodiment the vessel further comprises one or more therapeutic agents, agents useful for wound healing, and/or protein synthesis inhibitors. In another embodiment, the article of manufacturer further comprises a second vessel comprising one or more of the following: therapeutic agent, agent useful for wound healing, and/or protein synthesis inhibitor (e.g. steroid or Vitamin E).

A better understanding of the invention will be gained by reference to the following experimental section. The following experiments are illustrative and are not intended to limit the invention or the claims in any way.

### EXAMPLES

### Example 1

### Inhibition of Scar Formation in a Mouse Model

Methods of sequentially administering anti-connexin 43 polynucleotide preparation prepared with the following exemplary sequences: GTA ATT GCG GCA GGA GGA ATT GTT TCT CTC (connexin 43) (SEQ.ID.NO:2) and GAC AGA AAC AAT TCC TCC TGC CGC ATT TAC (sense control) (SEQ.ID.NO:7) are evaluated for the efficacy in the treatment of abnormal or excessive scarring.

Full thickness mouse wounds are made in adult mice, the majority of whom are six to eight weeks old and some of whom are fourteen to sixteen weeks old. Mice are pretreated for sixty days with anti-connexin polynucleotide, then wounds are made, and healing monitored. Mice are treated with a desired dose of an anti-connexin polynucleotide, e.g., an anti-connexin 43 polynucleotide, administered subcutaneously every other day.

Histological micrographs of open mouse wounds harvested at 7, 12, and 17 days post excision are made. The biopsies are fixed, embedded, sectioned and stained with hematoxylin and eosin.

The harvested wound tissue is examined to assess the effect of anti-connexin polynucleotide or scar formation. Density of blood vessels and granulation tissue in treated animals is examined compared to untreated controls. Mesenchymal cell infiltration is examined in treated compared to untreated animals. At 12 days, the open wounds in the controls are examined to assess degree of re-epithelialization and density of patent vessels, compared to the treated wound. In addition, the density of mesenchymal cells in treated granulation tissue is examined in the treated animals and in the controls. At 17 days, degree of closing is observed in both treated and untreated mouse wounds. The density of blood vessels is examined in the untreated mice, compared to the treated mice. In contrast, at day 17 after wounding, the density of mesenchymal cells and the thickness of the epidermis is observed in the treated mice and untreated mice. Thicker epidermis and greater density of mesenchymal cells would indicate retarded scar maturation.

### Example 2

### Inhibition of Scarring During Wound Healing

Anti-connexin polynucleotides, e.g., anti-connexin 43 polynucleotides, in the prevention of excessive scarring may be evaluated using a mouse model.

Mice are treated essentially the same as described in Example 1.

Endogenous synthesis of basic fibroblast growth factor in the wound is observed in treated and control of mice.

Histological analysis of the wounds in the control and treated mice compared contraction of full thickness wounds in mice treated with anti-connexin polynucleotide every other day after the wound is made, with untreated mice. The effect of treatment with anti-connexin polynucleotide once, and with repeated applications every other day after the wound is made on delay in the complete contraction of the wound and scarring is observed.

Breaking strength of linear scars after systemic administration of anti-connexin polynucleotide is observed at post wound day 7 and on post wound day 12, and optimally on day 40. The effect on wounds and scar formation of anti-connexin polynucleotide given on post wound days 0, 2, 4 or post wound days 0, 2, 4, 6, 8, and 10 is observed.

### Example 3

### Studies of the Effect of Anti-Connexin Polynucleotide in Conjunction with a Glucocorticoid on Human Keloid and Hypertrophic Scars

Subjects to be tested are those subjects with intractable keloid scars that had failed to respond to multiple therapeutic trials with glucocortoids (Kenalog™).

In order to determine if the anti-connexin polynucleotide can induce breakdown of the scar matrix and produce macroscopic shrinkage and softening of the scar, three subjects are given 1-50 micrograms or more of an anti-connexin polynucleotide, e.g., an anti-connexin 43 polynucleotide, in one lesion and 1 mM lidocaine in a similar lesion in the same or contralateral area of the body.

After treatment with anti-connexin polynucleotide or lidocaine the scars are observed for softening of the scars. The response of keloid scars to subsequent bi-weekly injection is observed. In subjects with hypertrophic scars, the response to anti-connexin polynucleotide therapy is also observed with regard to further softening and fading of the scars.

The effect of anti-connexin polynucleotides in subjects with burn scar is also observed using this protocol.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various embodiments and/or preferred embodiments and optional features, any and all modifications and variations of the concepts herein disclosed that may be resorted to by those skilled in the art are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and applicants reserve the right to revise the application or claims to refer specifically to any individual member or any subgroup of members of the Markush group.

Other embodiments are within the following claims. The patent may not be interpreted to be limited to the specific examples or embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

What is claimed is:

1. A method of treating a patient having an abnormal scar, which method comprises: (a) excising the abnormal scar, and (b) administering an anti-connexin polynucleotide to the patient in a quantity sufficient to prevent or reduce abnormal scarring at the site of the excision.

2. The method of claim 1, wherein the anti-connexin polynucleotide decreases connexin protein expression, wherein said connexin is selected from the group consisting of connexin 26, connexin 30, connexin 30.3, connexin 31.1, connexin 32, connexin 36, connexin 37, connexin 40, connexin 40.1, connexin 43, connexin 45, connexin 46 and connexin 46.6.

3. The method of claim 2 wherein the anti-connexin polynucleotide is an antisense oligonucleotide.

4. A method according to claim 3 wherein the antisense oligonucleotide decreases expression of connexin 43.

5. The method of claim 2 wherein the anti-connexin polynucleotide is an siRNA oligonucleotide or an RNAi oligonucleotide.

6. A method according to claim 1, wherein the abnormal scar is present in or on the skin.

7. A method according to claim 1, wherein the abnormal scar is present in or on the eye.

8. A method according to claim 1 or 4, wherein the scar is a keloid scar.

9. A method according to claim 1 or 4, wherein the scar is a hypertrophic scar.

10. A method according to claim 1 or 4, wherein the scar is an atrophic scar.

11. A method according to claim 1 or 4, wherein the scar is a widespread scar.

12. A method according to claim 1, wherein the anti-connexin polynucleotide inhibits intercellular communication by decreasing gap junction formation.

13. A method according to claim 1, wherein the anti-connexin polynucleotide inhibits intercellular communication by decreasing connexin 43 gap junction formation.

14. A method according to claim 2, wherein the connexin is a human connexin.

15. A method according to claim 2, wherein the connexin is human connexin 43.

16. A method of preventing or decreasing abnormal scar formation in a patient undergoing a surgical procedure, said method comprising administering a therapeutically effective amount of an anti-connexin polynucleotide to said patient.

17. A method according to claim 16 or wherein the anti-connexin polynucleotide is administered to an excision or incision.

18. A method according to claim 16 wherein the anti-connexin polynucleotide is administered to a debridement.

19. A method according to claim 16 wherein the anti-connexin polynucleotide is administered in the form of a liquid, gel, foam or spray.

20. A method according to claim 16 wherein said patient is at risk for keloid formation.

21. A method according to claim 16 wherein said patient is at risk for hypertrophic scar formation.

22. A method according to claim 16 wherein said patient is at risk for atrophic scar, or widespread scar formation.

23. A method according to claim 16, wherein the anti-connexin polynucleotide is an anti-connexin oligonucleotide.

24. A method according to claim 16 wherein the anti-connexin polynucleotide is oligonucleotide is selected from the group consisting of SEQ. I.D. NOS 1 to 12.

25. A method according to claim 16 wherein the connexin oligonucleotide is selected from SEQ. I.D. NOS. 1 and 2.

26. A method according to claim 16 wherein said anti-connexin polynucleotide is implanted or instilled.

27. A method according to any of claims 16-25 wherein the anti-connexin polynucleotide is an anti-connexin 43 compound.

28. A method according to claim 27 wherein said anti-connexin polynucleotide is administered topically.

29. A method to testing the activity of an anti-connexin polynucleotide for the prevention or reduction abnormal scarring, comprising contacting cells at risk of forming an abnormal scar with an anti-connexin polynucleotide, and determining or measuring the abnormal scarring prevention or reduction activity of said anti-connexin polynucleotide.

30. A method according to claim 29 wherein said method is carried out in vitro.

31. A method according to claim 29 wherein said method is carried out in vivo.

32. A method according to claim 29 wherein said method is carried out to test the activity of an anti-connexin polynucleotide to prevent or reduce keloid scar formation.

33. A method according to claim 29 wherein said method is carried out to test the activity of an anti-connexin polynucleotide to prevent or reduce hypertrophic scar formation.

34. A method according to claim 29 wherein said method is carried out to test the activity of an anti-connexin polynucleotide to prevent or reduce the formation of atrophic scars, or widespread scars.

35. A method according to claim 29 wherein said anti-connexin is an oligonucleotide.

36. An article of manufacture comprising: (a) a pharmaceutical composition having (i) a therapeutically effective amount of an anti-connexin polynucleotide, and (ii) a pharmaceutically acceptable carrier, and (b) instructions for administering the pharmaceutical composition to a patient having or at risk of having an abnormal scar.

37. The article of claim 36 wherein the instructions describe administration of the pharmaceutical composition to the patient to treat an abnormal scar by excising the abnormal scar and administering the pharmaceutical composition in a quantity sufficient to prevent or reduce abnormal scarring at a site of the excision.

38. The article of claim 36 wherein the abnormal scar is selected from the group consisting of keloid scars, hypertrophic scars, atrophic scars, and widespread scars.

39. The article of claim 36 wherein the anti-connexin is an oligonucleotide.

40. The article of claim 39 wherein the oligonucleotide is an anti-connexin 43 oligonucleotide.

41. A method of making an article of manufacture, which method comprises: combining (a) a container including a pharmaceutical composition comprising (i) an anti-connexin polynucleotide, and (ii) a pharmaceutically acceptable carrier, and (b) labeling instructions for treating a patient having an abnormal scar by administering the pharmaceutical composition to a patient having an abnormal scar.

42. A method according to claim 41 wherein the instructions describe administration of the pharmaceutical composition to the patient to treat an abnormal scar by excising the abnormal scar and administering the pharmaceutical composition in a quantity sufficient to prevent or reduce abnormal scarring at a site of the excision.

43. A method according to claim 41 wherein the abnormal scar is selected from the group consisting of keloid scars, hypertrophic scars, atrophic scars, and widespread scars.

44. The article of claim 41 wherein the anti-connexin is an oligonucleotide.

45. The article of claim 44 wherein the oligonucleotide is an anti-connexin 43 oligonucleotide.

46. A method of decreasing or preventing excessive scar formation which comprises administration to a subject in need of treatment an effective amount of an anti-connexin polynucleotide.

47. A method according to claim 46 wherein the anti-connexin polynucleotide decreases connexin protein expression.

48. A method according to claim 47 wherein the connexin is selected from the group consisting of connexin 26, connexin 30, connexin 30.3, connexin 31.1, connexin 32, connexin 36, connexin 37, connexin 40, connexin 40.1, connexin 43, connexin 45, connexin 46 and connexin 46.6.

49. A method according to claim 48 wherein the connexin is connexin 43.

50. A method according to claim 47 wherein the anti-connexin polynucleotide is an antisense oligonucleotide.

51. A method according to claim 50 wherein the antisense oligonucleotide is anti-connexin 43 oligonucleotide.

52. A method according to claim 50 wherein the oligonucleotide has a sequence selected from SEQ. ID. NOS. 1 to 12.

53. A method according to claim 50 wherein the oligonucleotide has a sequence selected from SEQ. ID. NOS. 1 and 2.

54. A method according to any of claims 46-53 wherein the anti-connexin polynucleotide decreases or prevents keloid formation of keloid scars, hypertrophic scars, atrophic scars, or widespread scars.

55. A method according to claim 46 wherein the anti-connexin polynucleotide is implanted or instilled.

56. A method according to claim 55 wherein the anti-connexin polynucleotide is an anti-connexin 43 polynucleotide.

57. A method according to claim 56 wherein the anti-connexin 43 polynucleotide is an antisense oligonucleotide.

58. A method according to claim 57 wherein the antisense oligonucleotide is an anti-connexin 43 antisense oligonucleotide.

59. A method according to claim 46 wherein said anti-connexin polynucleotide is administered topically.

60. A method according to claim 59 wherein the anti-connexin polynucleotide is an anti-connexin 43 polynucleotide.

61. A method according to claim 60 wherein the anti-connexin 43 polynucleotide is an antisense oligonucleotide.

62. A method according to claim 61 wherein the antisense oligonucleotide is an anti-connexin 43 antisense oligonucleotide.

63. The method of any of claim 1, 16, 41 or 46, wherein the patient or subject is a human.

64. The method of any of claim 1, 16, 41 or 46, wherein the patient or subject is a non-human animal.

65. The method of claim 64, wherein the non-human animal is a sports or pet animal.

66. The method of claim 65, wherein the sports or pet animal is a horse, a dog or a cat.

67. The article of manufacture of claim 36, wherein the patient is a human.

68. The article of manufacture of claim 36, wherein the patient is a non-human animal.

69. The article of manufacture of claim 68, wherein the non-human animal is a sports or pet animal.

70. The article of manufacture of claim 69, wherein the sports or pet animal is a horse, a dog or a cat.


SEQUENCE LISTING (12 sequences)

SEQ ID NO: 1 - DNA, 30 bp, Artificial
  Mammalian-derived
gtaattgcggcaagaagaattgtttctgtc

SEQ ID NO: 2 - DNA, 30 bp, artificial
  Mammalian-derived
gtaattgcggcaggaggaattgtttctgtc

SEQ ID NO: 3 - DNA, 30 bp, artificial
  Mammalian-derived
ggcaagagacaccaaagacactaccagcat

SEQ ID NO: 4 - DNA, 27 bp, artificial
  Mammalian-derived
tcctgagcaatacctaacgaacaaata

SEQ ID NO: 5 - DNA, 20 bp, artificial
  Mammalian-derived
catctccttggtgctcaacc

SEQ ID NO: 6 - DNA, 20 bp, artificial
  Mammalian-derived
ctgaagtcgacttggcttgg

SEQ ID NO: 7 - DNA, 21 bp, Artificial
  Mammalian-derived
ctcagatagtggccagaatgc

SEQ ID NO: 8 - DNA, 20 bp, artificial
  Mammalian-derived
ttgtccaggtgactccaagg

SEQ ID NO: 9 - DNA, 25 bp, artificial
  Mammalian-derived
cgtccgagcccagaaagatgaggtc

SEQ ID NO: 10 - DNA, 19 bp, artificial
  Mammalian-derived
agaggcgcacgtgagacac

SEQ ID NO: 11 - DNA, 19 bp, artificial
  Mammalian-derived
tgaagacaatgaagatgtt

SEQ ID NO: 12 - DNA, 25 bp, artificial
  Mammalian-derived
tttcttttctatgtgctgttggtga